(19) United States
(12) Patent Application Publication
(10) Pub. No.: US 2007/0123453 A1
(43) Pub. Date: May 31, 2007

(54) Uses of melanocortin-4 receptor (mc4r) agonist peptides administered by continuous infusion

(76) Inventors: Mark Louis Heiman, Indianapolis, IN (US); JeAnne L. Hertel, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 10/588,667

(22) PCT Filed: Jun. 17, 2004
(86) PCT No.: PCT/US04/16623

§ 371 Date: Aug. 4, 2006

Related U.S. Application Data
(60) Provisional application No. 60/557,347, filed on Mar. 29, 2004.
(60) Provisional application No. 60/570,676, filed on May 13, 2004.

(51) Int. Cl. A61K 38/12

(52) U.S. Cl. 514/9

(57) ABSTRACT

The present invention provides a method of inducing weight loss in a patient, comprising continuous infusion of an effective amount of an MC4R agonist peptide into the patient. Additionally, the present invention provides a method of treating obesity in a patient, comprising continuous infusion of an effective amount of an MC4R agonist peptide into the patient. Furthermore, the present invention provides the use of an MC4R agonist peptide for the manufacture of a medicament for the treatment of obesity, wherein the medicament is administered by continuous infusion.

The melanocortin-4 receptor (MC4R) is a G-protein coupled receptor (GPCR). MC4R mediates a signal that it receives from the endogenous melanocortin stimulating hormones (MSH) and the agouti related protein peptide (AGRP) in the hypothalamus. The former peptides are processed from a proopiomelanocortin (POMC) precursor protein produced by the neurons in the arcuate nucleus of the hypothalamus. Those peptides are competitive full agonists for the MC4 receptor. Conversely, AGRP is reported to be either a competitive antagonist or an inverse agonist at the same receptor. This endogenous messenger is also produced and released by neurons in the hypothalamus but distinct from those synthesizing POMC. Together, the melanocortin system is part of the neuronal hypothalamic network regulating energy balance.

It has been proposed that during physiological states characterized by a negative energy balance, AGRP signaling is enhanced and POMC signaling is reduced. Further, those responses are thought to participate in correcting the negative energy balance. Specifically, AGRP signaling would dominate over MSH signaling, resulting in enhanced appetite and decreased energy expenditure via decreased activity of the sympathetic nervous system.

Etiology and pathophysiology of obesity remains a subject of intense study. There are rare examples of obese individuals and obese rodents with mutations of MC4R or POMC genes. Over-expression of an AGRP transgene will also present an obese mouse. There are no examples of over-expression of POMC producing a lean phenotype. This raises the possibility that MC4R may be desensitized during continuous exposure to its agonists. Indeed, there are many examples of GPCRs that are down regulated by chronic exposure to their agonists.

Daily peripheral administration of the MSH agonist melanotan II (MT-II) for at least one week decreases weight gain in rodents, indicating that a peripheral injection of the peptide will trigger the MC4 receptor in the hypothalamus and that a lean phenotype can be realized. Further, such studies suggest no desensitization after intermittent administration. Because those peptides have a short half-life and were only administered intermittently, it follows that the receptor was also only infrequently occupied and that may have prevented any down regulation or desensitization.

A need exists to find an agonist capable of triggering the MC4 receptor, capable of being administered such that the receptor remains occupied, but without down regulation or desensitization of the receptor. Meeting this need will provide a means to induce weight loss and overcome obesity, a disease that has major debilitating effects on the body.

The present invention provides a method of inducing weight loss in a patient, comprising continuous infusion of an effective amount of an MC4R agonist peptide into the patient. Additionally, the present invention provides a method of treating obesity in a patient, comprising continuous infusion of an effective amount of an MC4R agonist peptide into the patient. Furthermore, the present invention provides the use of an MC4R agonist peptide for the manufacture of a medicament for the treatment of obesity, wherein the medicament is administered by continuous infusion.

The instant invention demonstrates that when the same mass of an MC4R agonist peptide is delivered to patients using two different methods: (1) a single daily bolus subcutaneous administration, or (2) by continuous subcutaneous infusion, the peptide is much more effective when administered continuously than intermittently. Those data suggest that the MC4 receptor can be continuously occupied with an agonist without down regulation or desensitization.

Moreover, a low rate of infusion, for example approximately 2 μg/hr of Ac-D-Arg-cyclo[Cys-Glu-His-D-Phe-Arg-Trp-Cys]-NH₂ infused into the subcutaneous environment, is sufficient to overcome metabolism and dilution of the peptides to successfully bind the hypothalamic receptor in quantities that would overcome competition by AGRP.

Furthermore, delivery of the peptide via continuous infusion allows the MC4 receptor to remain continuously occupied. Importantly, this overcomes problems associated with bolus injections. For instance, due to short half-life of the MC4R agonist peptide, shortly after a bolus injection is made, the peptide degrades, leaving the receptor open for antagonists or inverse agonists to occupy. Occupation by an antagonist or inverse agonist may not induce weight loss; conversely, it may induce weight gain. Yet, with continuous infusion of the MC4R agonist peptide, the receptor remains occupied with the agonist. Additionally, potential side effects caused by bolus injections, such as penile erection, may be avoided.

For the purposes of the present invention, as disclosed and claimed herein, the following terms are as defined below.

“Continuous infusion” of an MC4R agonist peptide refers to controlled parenteral delivery of the peptide to a patient for an extended period of time. Administration of the peptide may be accomplished by, but is not limited to, delivery via pump, depot, suppository, pessary, transdermal patch or other topical administration (such as buccal, sublingual, spray, ointment, creme, or gel) using, for example, subcutaneous, intramuscular, intraperitoneal, intravenous, intracerebral, or intraarterial administration.

A pump delivering the MC4R agonist peptide into the body may be implanted in the patient's body. Alternatively, the patient may wear a pump externally, being attached to the patient's body via catheter, needle, or some other connective means. Any pump that is suitable for the delivery of pharmaceuticals to a patient may be used. Examples include pumps such as those disclosed in U.S. Pat. No. 6,659,982.

A depot is a biocompatible polymer system containing the MC4R agonist peptide and delivering the peptide over time. Examples include microspheres, microcapsules, nanoparticles, liposomes, a hydrogel, or other polymeric implants. Preferred periods for delivery of agonist by depot include one week, two weeks, and one month periods. If needed, another depot will be delivered to the patient for continued delivery of peptide.

Engineering the MC4R agonist peptide to have a prolonged half-life will also result in continuous delivery of the MC4 receptor agonist to the receptor. Such modifications include conjugations with larger proteins such as albumin, antibody and antigen or chemical modifications that may increase half-life by linking fatty acids, polyethylene glycol (PEG) polymers, and other agents.

An “MC4R agonist peptide” utilized in the instant invention includes any agonist peptide which has affinity for the MC4 receptor. Examples include, but are not limited to, MC4R agonists disclosed in the following art: U.S. Pat. No. 5,674,839; WO 01/52880; WO 03/006604; WO 00/36136; WO 01/00224; WO 01/13112; WO 00/58361; U.S. Pat. No. 6,613,874; WO 02/26774; WO 99/54358; WO 01/74844; WO 02/18437; WO 98/27113; WO 01/05401; U.S. Pat. No. 5,731,408; and WO 01/85930, which are herein incorporated by reference.

In another embodiment, the MC4R agonist peptide for use in the present invention is represented by the following Structural Formula I (SEQ ID NO:199):

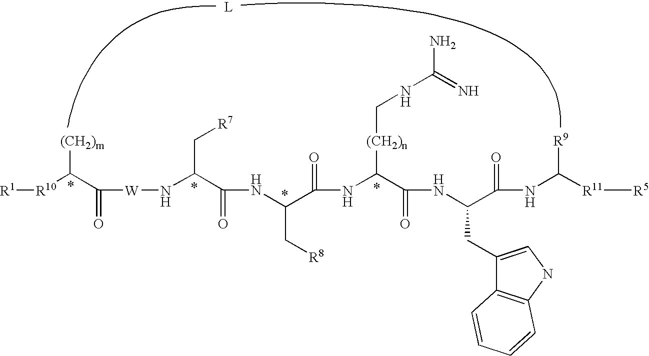

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 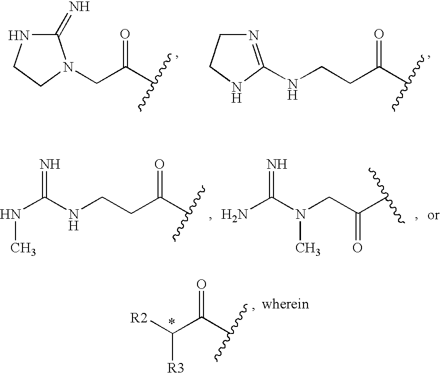
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr—NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 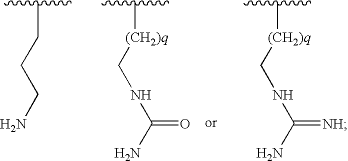
q is 0, 1, 2, or 3;
R⁶ is a phenyl or C₈-C₁₄ bicyclic aryl;
m is 1 or 2;
n is 1, 2, 3, or 4;
R⁹ is (CH₂)ₚ or (CH₃)₂C—;
p is 1 or 2;
R¹⁰ is NH— or is absent;
R⁷ is a 5- or 6-membered heteroaryl or a 5- or 6-membered heteroaryl ring optionally substituted with R⁴;
R⁴ is H, C₁-C₄ straight or branched alkyl, phenyl, benzyl, or (C₆H₅)—CH₂—O—CH₂—;
R⁸ is phenyl, a phenyl ring optionally substituted with X, or cyclohexyl;
X is H, Cl, F, Br, methyl, or methoxy;
R¹¹ is —C(O) or —CH₂;
R⁵ is —NH₂, —OH, glycinol, NH₂-Pro-Ser-, NH₂-Pro-Lys-, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl; and
L is —S—S— or —S—CH₂—S—.

A preferred group of MC4R agonist peptides for use in the present invention includes compounds of Structural Formula II (SEQ ID NO:200):

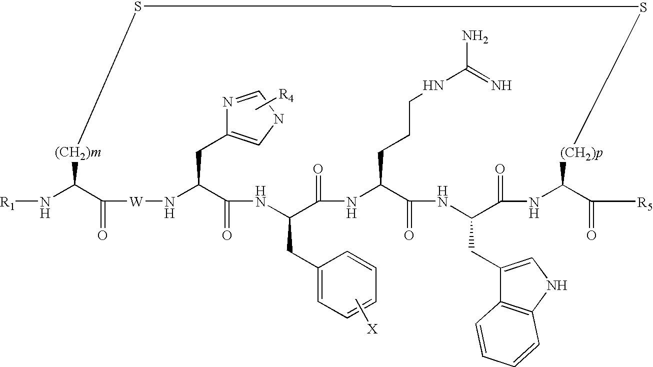

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 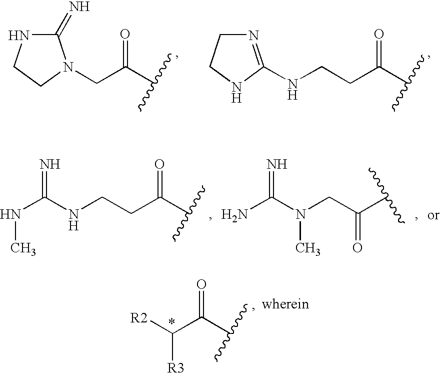
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 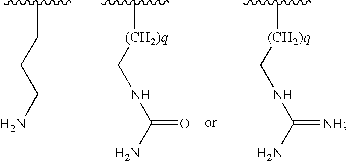
q is 0, 1, 2, or 3;
R⁶ is a phenyl or C₈-C₁₄ bicyclic aryl;
m is 1 or 2;
p is 1 or 2;
R⁴ is H, C₁-C₄ straight or branched alkyl, phenyl, benzyl, or (C₆H₅)—CH₂—O—CH₂—;
X is H, Cl, F, Br, methyl, or methoxy; and
R⁵ is —NH₂, —OH, glycinol, NH₂-Pro-Ser-, NH₂-Pro-Lys, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl.

Another preferred group of MC4R agonist peptides for use in the present invention are compounds of the Structural Formula II, wherein W is Glu or is absent; R₄ is H or CH₃; X is H, Cl, F, or Br; and R₅ is NH₂ or OH.

Yet another preferred group of MC4R agonist peptides are compounds of Structural Formula II wherein W is Glu or is absent; R¹ is H-, Ac-, Arg-, Ac-Arg-, or Ac-D-Arg-; m is 1 or 2; p is 1; and R⁵ is NH₂ or OH.

A preferred compound for use in the present invention is an MC4R agonist peptide of Structural Formula II wherein W is absent; R¹ is Ac-; m is 2; p is 1; and R⁵ is NH₂.

Another preferred compound for use in the present invention is an MC4R agonist peptide of Structural Formula II wherein W is Glu; R¹ is Ac-Arg-; m is 1; p is 1; and R⁵ is NH₂.

Another preferred compound for use in the present invention is an MC4R agonist peptide of Structural Formula II wherein W is absent; R¹ is H; m is 2; p is 1; and R⁵ is NH₂.

Another preferred compound for use in the present invention is an MC4R agonist peptide of Structural Formula II wherein W is absent; R¹ is Arg-; m is 2; p is 1; and R⁵ is OH.

A most preferred compound for use in the present invention is an MC4R agonist peptide of Structural Formula II wherein W is Glu; R¹ is Ac-D-Arg-; m is 1; p is 1; and R⁵ is NH₂.

An alternative preferred group of MC4R agonist peptides for use in the present invention is represented by the following Structural Formula III (SEQ ID NO:201):

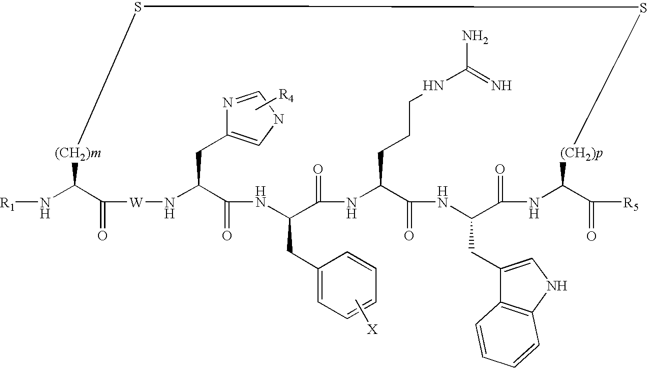

and pharmaceutically acceptable salts thereof, wherein
W is a single bond, Glu, Gln, Asp, Asn, Ala, Gly, Thr, Ser, Pro, Met, Ile, Val, Arg, His, Tyr, Trp, or Phe;
R₁ is —H, —C(O)CH₃, —C(O)(CH₂)₁₋₄NH-C(NH)NH₂, Tyr-βArg, gluconoyl-Tyr-Arg, Ac-Dab, Ac-Dap, N-succinyl-Tyr-Arg, N-propionyl, N-valeryl, N-glutaryl-Tyr-Arg, N-butyryl, 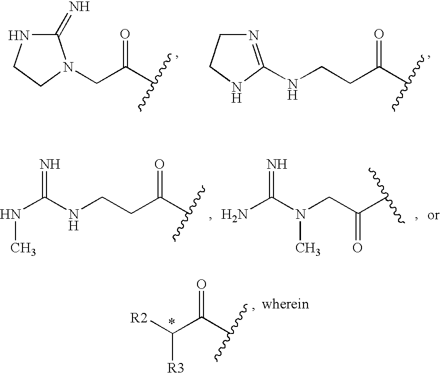
R₂ is —H, —NH₂, —NHC(O)CH₃, —NHC(O)(CH₂)₁₋₄CH₃, Tyr, or —NH-Tyr-C(O)CH₃;
R₃ is C₁-C₄ straight or branched alkyl, Ser, Ile, Arg, 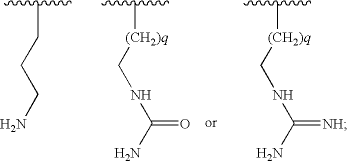
q is 0, 1, 2, or 3;
m is 1 or 2;
p is 1 or 2;
R₄ is —H, —CH₃, or —(CH₂)₁₋₃CH₃;
X is —H, —Cl, —F, —Br, methyl, or methoxy; and
R₅is —NH₂, —OH, glycinol, -Ser-Pro-NH₂, -Lys-Pro-NH₂, -Ser-OH, -Ser-Pro-OH, -Lys-Pro-OH, -Arg-Phe-NH₂, -Glu-NH₂, —NHR, or —OR, where R is —CH₃ or —(CH₂)₁₋₃CH₃.

MC4R agonist peptides for use in the present invention include, but are not limited to, those compounds listed in the following table:

TABLE 1
Specific compounds within the present invention.

| No. | Name |
|---|---|
| 1 | Ac-cyclo[Cys-His-D-Phe-Arg-Trp-Cys]-NH₂ |
| 2 | Ac-Cya-Arg-cyclo[Cys-Ala-His-D-Phe-Arg-Trp-Cys]-NH₂ |
| 3 | Ac-Tyr-Arg-cyclo[Cys-Ala-His-D-Phe-Arg-Trp-Cys]-NH₂ |
| 4 | Ac-Tyr-Arg-cyclo[Cys-Arg-His-D-Phe-Arg-Trp-Cys]-NH₂ |
| 5 | Ac-Tyr-Arg-cyclo[Cys-Asn-His-D-Phe-Arg-Trp-Cys]-NH₂ |
| 6 | Ac-cyclo[Cys-Asp-His-D-Phe-Arg-Trp-Cys]-NH₂ |
| 7 | Ac-Tyr-Arg-cyclo[Cys-Asp-His-D-Phe-Arg-Trp-Cys]-NH₂ |
| 8 | Ac-cyclo[Cys-Gln-His-D-Phe-Arg-Trp-Cys]-NH₂ |
| 9 | Ac-Tyr-Arg-cyclo[Cys-Gln-His-D-Phe-Arg-Trp-Cys]-OH |
| 10 | Ac-Tyr-Arg-cyclo[Cys-Gln-His-D-Phe-Arg-Trp-Cys]-OMe |
| 11 | Tyr-Arg-cyclo[Cys-Gly-His-D-Phe-Arg-Trp-Cys]-NH₂ |
| 12 | Ac-Tyr-Arg-cyclo[Cys-Gly-His-D-Phe-Arg-Trp-Cys]-NH₂ |
| 13 | Ac-Tyr-Arg-cyclo[Cys-His-His-D-Phe-Arg-Trp-Cys]-NH₂ |
| 14 | Ac-Tyr-Arg-cyclo[Cys-Ile-His-D-Phe-Arg-Trp-Cys]-NH₂ |
| 15 | Ac-cyclo[Cys-Leu-His-D-Phe-Arg-Trp-Cys]-NH₂ |
| 16 | Ac-cyclo[Cys-Lys-His-D-Phe-Arg-Trp-Cys]-NH₂ |
| 17 | N-methyl-Tyr-Arg-cyclo[Cys-Met-His-D-Phe-Arg-Trp-Cys]-NH₂ |
| 18 | Ac-Tyr-Arg-cyclo[Cys-Met-His-D-Phe-Arg-Trp-Cys]-NH₂ |
| 19 | Ac-Tyr-Arg-cyclo[Cys-Phe-His-D-Phe-Arg-Trp-Cys]-NH₂ |
| 20 | Ac-Tyr-Arg-cyclo[Cys-Pro-His-D-Phe-Arg-Trp-Cys]-NH₂ |
| 21 | Ac-Tyr-Arg-cyclo[Cys-Ser-His-D-Phe-Arg-Trp-Cys]-NH₂ |
| 22 | Ac-Tyr-Arg-cyclo[Cys-Thr-His-D-Phe-Arg-Trp-Cys]-NH₂ |
| 23 | Ac-Tyr-Arg-cyclo[Cys-Trp-His-D-Phe-Arg-Trp-Cys]-NH₂ |
| 24 | Ac-Tyr-Arg-cyclo[Cys-Tyr-His-D-Phe-Arg-Trp-Cys]-NH₂ |
| 25 | Ac-Tyr-Arg-cyclo[Cys-Val-His-D-Phe-Arg-Trp-Cys]-NH₂ |
| 26 | Ac-Arg-cyclo[Cys-Cya-His-D-Phe-Arg-Trp-Cys]-NH₂ |
| 27 | Ac-D-Arg-cyclo[Cys-Cya-His-D-Phe-Arg-Trp-Cys]-NH₂ |
| 28 | Ac-Tyr-Arg-cyclo[Cys-Cya-His-D-Phe-Arg-Trp-Cys]-NH₂ |
| 29 | cyclo[Cys-Glu-His-D-Phe-Arg-Trp-Cys]-NH₂ |
| 30 | Ac-cyclo[Cys-Glu-His-D-Phe-Arg-Trp-Cys]-NH₂ |
| 31 | Ac-cyclo[Cys-Glu-His-(4-F-D-Phe)-Arg-Trp-Cys]-NH₂ |
| 32 | Ac-cyclo[Cys-Glu-His-(4-Cl-D-Phe)-Arg-Trp-Cys]-NH₂ |
| 33 | Ac-cyclo[Cys-Glu-His-(4-Br-D-Phe)-Arg-Trp-Cys]-NH₂ |
| 34 | Ac-cyclo[Cys-Glu-(1-Me-His)-D-Phe-Arg-Trp-Cys]-NH₂ |
| 35 | Ac-cyclo[Cys-Glu-His-D-Phe-Arg-Trp-Cys]-Lys-Pro-NH₂ |
| 36 | Ac-cyclo[Cys-Glu-His-D-Phe-Arg-Trp-Cys]-Ser-Pro-NH₂ |
| 37 | N-propionyl-cyclo[Cys-Glu-His-D-Phe-Arg-Trp-Cys]-NH₂ |
| 38 | N-butyryl-cyclo[Cys-Glu-His-D-Phe-Arg-Trp-Cys]-NH₂ |
| 39 | N-valeryl-cyclo[Cys-Glu-His-D-Phe-Arg-Trp-Cys]-NH₂ |
| 40 | 3-guanidinopropionyl-cyclo[Cys-Glu-His-D-Phe-Arg-Trp-Cys]-NH₂ |
| 41 | 4-guanidinobutyryl-cyclo[Cys-Glu-His-D-Phe-Arg-Trp-Cys]-NH₂ |
| 42 | 5-guanidinovaleryl-cyclo[Cys-Glu-His-D-Phe-Arg-Trp-Cys]-NH₂ |
| 43 | Ac-diaminopropionyl-cyclo[Cys-Glu-His-D-Phe-Arg-Trp-Cys]-NH₂ |
| 44 | Ac-diaminobutyryl-cyclo[Cys-Glu-His-D-Phe-Arg-Trp-Cys]-NH₂ |
| 45 | Arg-cyclo[Cys-Glu-His-D-Phe-Arg-Trp-Cys]-OH |
| 46 | D-Arg-cyclo[Cys-Glu-His-D-Phe-Arg-Trp-Cys]-NH₂ |
| 47 | Ac-D-Arg-cyclo[Cys-Glu-His-Phe-Arg-Trp-Cys]-NH₂ |
| 48 | Ac-Arg-cyclo[Cys-Glu-His-D-Phe-Arg-Trp-Cys]-NH₂ |
| 49 | Ac-Arg-cyclo[Cys-Glu-His-D-Phe-Arg-Trp-Cys]-OH |
| 50 | Ac-Arg-cyclo[Cys-Glu-His-(4-Cl-D-Phe)-Arg-Trp-Cys]-NH₂ |
| 51 | Ac-Arg-cyclo[Cys-Glu-(1-Me-His)-D-Phe-Arg-Trp-Cys]-NH₂ |
| 52 | Ac-D-Arg-cyclo[Cys-Glu-His-D-Phe-Arg-Trp-Cys]-NH₂ |
| 53 | Ac-D-Arg-cyclo[Cys-Glu-His-D-Phe-Arg-Trp-Cys]-OH |
| 54 | Ac-hArg-cyclo[Cys-Glu-His-D-Phe-Arg-Trp-Cys]-NH₂ |
| 55 | Ac-Cit-cyclo[Cys-Glu-His-D-Phe-Arg-Trp-Cys]-NH₂ |
| 56 | Ac-Cit-cyclo[Cys-Glu-(1-Me-His)-D-Phe-Arg-Trp-Cys]-NH₂ |
| 57 | Ac-Leu-cyclo[Cys-Glu-His-D-Phe-Arg-Trp-Cys]-NH₂ |
| 58 | Ac-Lys-cyclo[Cys-Glu-His-D-Phe-Arg-Trp-Cys]-NH₂ |
| 59 | Ac-Lys(ipr)-cyclo[Cys-Glu-His-D-Phe-Arg-Trp-Cys]-NH₂ |
| 60 | Ac-nLeu-cyclo[Cys-Glu-His-D-Phe-Arg-Trp-Cys]-NH₂ |
| 61 | Ac-nLeu-cyclo[Cys-Glu-His-D-Phe-Arg-Trp-Cys]-Ser-Pro-NH₂ |
| 62 | Ac-Orn-cyclo[Cys-Glu-His-D-Phe-Arg-Trp-Cys]-NH₂ |
| 63 | Ac-Val-cyclo[Cys-Glu-His-D-Phe-Arg-Trp-Cys]-NH₂ |
| 64 | N-(2-naphthalenesulfonyl)-D-Arg-cyclo[Cys-Glu-His-D-Phe-Arg-Trp-Cys]-NH₂ |
| 65 | N-(2-naphthalenesulfonylamino-4-oxo-butyryil)-D-Arg-cyclo[Cys-Glu-His-D-Phe-Arg-Trp-Cys]- |
| | NH₂ |
| 66 | 3-(4-hydroxyphenyl)propionyl-Arg-cyclo[Cys-Glu-His-D-Phe-Arg-Trp-Cys]-NH₂ |
| 67 | 3-(4-methylbenzoyl)propionyl-Arg-cyclo[Cys-Glu-His-D-Phe-Arg-Trp-Cys]-NH₂ |
| 68 | Tyr-Arg-cyclo[Cys-Glu-His-D-Phe-Arg-Trp-Cys]-NH₂ |
| 69 | Tyr-Arg-cyclo[Cys-Glu-His-D-Phe-Arg-Trp-Cys]-OH |
| 70 | Tyr-Arg-cyclo[Cys-Glu-His-D-Phe-Arg-Trp-Cys]-NH-(CH₂)₆-NH₂ |
| 71 | Tyr-Arg-cyclo[Cys-Glu-His-D-Phe-Arg-Trp-Cys]-Glu-NH₂ |
| 72 | Ac-Tyr-Arg-cyclo[Cys-Glu-His-D-Phe-Arg-Trp-Cys]-NH₂ |
| 73 | Ac-Tyr-Arg-cyclo[Cys-Glu-His-D-Phe-Arg-Trp-Cys]-OH |
| 74 | N-succinyl-Tyr-Arg-cyclo[Cys-Glu-His-D-Phe-Arg-Trp-Cys]-NH₂ |
| 75 | N-glutaryl-Tyr-Arg-cyclo[Cys-Glu-His-D-Phe-Arg-Trp-Cys]-NH₂ |
| 76 | N-glutaryl-Tyr-Arg-cyclo[Cys-Glu-His-D-Phe-Arg-Trp-Cys]-OH |
| 77 | gluconoyl-Tyr-Arg-cyclo[Cys-Glu-His-D-Phe-Arg-Trp-Cys]-NH₂ |
| 78 | Ac-Tyr-Arg-cyclo[Cys-Glu-His-D-Phe-Arg-Trp-Cys] alcohol |
| 79 | Ac-Tyr-D-Arg-cyclo[Cys-Glu-His-D-Phe-Arg-Trp-Cys]-NH₂ |
| 80 | Ac-Tyr-Arg-cyclo[D-Cys-Glu-His-D-Phe-Arg-Trp-Cys]-NH₂ |
| 81 | Ac-Tyr-Arg-cyclo[Cys-Glu-(1-Me-His)-D-Phe-Arg-Trp-Cys]-NH₂ |
| 82 | Ac-Tyr-Arg-cyclo[Cys-Glu-(1-Me-D-His)-D-Phe-Arg-Trp-Cys]-NH₂ |
| 83 | Ac-Tyr-Arg-cyclo[Cys-Glu-His-(4-F-D-Phe)-Arg-Trp-Cys]-NH₂ |
| 84 | Ac-Tyr-Arg-cyclo[Cys-Glu-(1-Me-His)-(4-F-D-Phe)-Arg-Trp-Cys]-NH₂ |
| 85 | Ac-Tyr-Arg-cyclo[Cys-Glu-(1-Me-D-His)-(4-F-D-Phe)-Arg-Trp-Cys]-NH₂ |
| 86 | Ac-Tyr-Arg-cyclo[Cys-Glu-His-(4-Cl-D-Phe)-Arg-Trp-Cys]-NH₂ |
| 87 | Ac-Arg-cyclo[Cys-Glu-(1-Me-His)-(4-Cl-D-Phe)-Arg-Trp-Cys]-NH₂ |
| 88 | Ac-Tyr-Arg-cyclo[Cys-Glu-(1-Me-D-His)-(4-Cl-D-Phe)-Arg-Trp-Cys]-NH₂ |
| 89 | Ac-Tyr-Arg-cyclo[Cys-Glu-His-(4-Br-D-Phe)-Arg-Trp-Cys]-NH₂ |
| 90 | Ac-Tyr-Arg-cyclo[Cys-Glu-(1-Me-His)-(4-Br-D-Phe)-Arg-Trp-Cys]-NH₂ |
| 91 | Ac-Tyr-Arg-cyclo[Cys-Glu-(1-Me-D-His)-(4-Br-D-Phe)-Arg-Trp-Cys]-NH₂ |
| 92 | Ac-Tyr-Arg-cyclo[Cys-Glu-His-(4-Me-D-Phe)-Arg-Trp-Cys]-NH₂ |
| 93 | Ac-Tyr-Arg-cyclo[Cys-Glu-His-(4-OMe-D-Phe)-Arg-Trp-Cys]-NH₂ |
| 94 | Ac-Tyr-Arg-cyclo[Cys-Glu-(1-Me-His)-(4-OMe-D-Phe)-Arg-Trp-Cys]-NH₂ |
| 95 | Ac-Tyr-Arg-cyclo[Cys-Glu-(1-Me-D-His)-(4-OMe-D-Phe)-Arg-Trp-Cys]-NH₂ |
| 96 | Ac-Tyr-Arg-cyclo[Cys-Glu-(3-Me-His)-D-Phe-Arg-Trp-Cys]-NH₂ |
| 97 | Ac-Tyr-Arg-cyclo[Cys-Glu-(5-Me-His)-D-Phe-Arg-Trp-Cys]-NH₂ |
| 98 | Ac-Tyr-Arg-cyclo[Cys-Glu-(5-Me-D-His)-D-Phe-Arg-Trp-Cys]-NH₂ |
| 99 | Ac-Tyr-Arg-cyclo[Cys-Glu-(1-benzyl-His)-D-Phe-Arg-Trp-Cys]-NH₂ |
| 100 | Ac-Tyr-Arg-cyclo[Cys-Glu-(1-benzyl-D-His)-D-Phe-Arg-Trp-Cys]-NH₂ |
| 101 | Ac-Tyr-Arg-cyclo[Cys-Glu-(1-Bom-His)-D-Phe-Arg-Trp-Cys]-NH₂ |
| 102 | Ac-Tyr-Arg-cyclo[Cys-Glu-(1-pyrazolyl-Ala)-D-Phe-Arg-Trp-Cys]-NH₂ |
| 103 | Ac-Tyr-Arg-cyclo[Cys-Glu-(4-phenyl-1H-imidazol-2-yl-Ala)-D-Phe-Arg-Trp-Cys]-NH₂ |
| 104 | Ac-Tyr-Arg-cyclo[Cys-Glu-(4-phenyl-1H-imidazol-2-yl-D-Ala)-D-Phe-Arg-Trp-Cys]-NH₂ |
| 105 | Ac-Tyr-Arg-cyclo[Cys-Glu-(2-pyrazine-Ala)-D-Phe-Arg-Trp-Cys]-NH₂ |
| 106 | Ac-Tyr-Arg-cyclo[Cys-Glu-(β-(1,2,4-triazol-3-yl))-Ala)-D-Phe-Arg-Trp-Cys]-NH₂ |
| 107 | Ac-Tyr-Arg-cyclo[Cys-Glu-(β-(1,2,4-triazol-3-yl))-D-Ala)-D-Phe-Arg-Trp-Cys]-NH₂ |
| 108 | Ac-Tyr-Arg-cyclo[Cys-Glu-(β-((1-benzyl)-1,2,4-triazol-3-yl))-Ala)-D-Phe-Arg-Trp-Cys]-NH₂ |
| 109 | Ac-Tyr-Arg-cyclo[Cys-Glu-(β-((1-benzyl)-1,2,4-triazol-3-yl))-D-Ala)-D-Phe-Arg-Trp-Cys]-NH₂ |
| 110 | Ac-Tyr-Arg-cyclo[Cys-Glu-(β-(2-furyl)-Ala)-D-Phe-Arg-Trp-Cys]-NH₂ |
| 111 | Ac-Tyr-Arg-cyclo[Cys-Glu-(β-(thien-2-yl)-Ala)-D-Phe-Arg-Trp-Cys]-NH₂ |
| 112 | Ac-Tyr-Arg-cyclo[Cys-Glu-(β-(1,3-thiazol-4-yl)-Ala)-D-Phe-Arg-Trp-Cys]-NH₂ |
| 113 | Ac-Tyr-Arg-cyclo[Cys-Glu-(β-(pyridin-4-yl)-Ala)-D-Phe-Arg-Trp-Cys]-NH₂ |
| 114 | Ac-Tyr-Arg-cyclo[Cys-Glu-His-D-Phe-Arg-Trp-Cys]-glycinol |
| 115 | Ac-Tyr-Arg-cyclo[Cys-Glu-His-D-Phe-Arg-Trp-Cys]-2-(2-aminoethoxy)ethanol |
| 116 | Ac-Tyr-Arg-cyclo[Cys-Glu-His-D-Phe-Arg-Trp-Cys]-Ser alcohol |
| 117 | Ac-Tyr-Arg-cyclo[Cys-Glu-His-D-Phe-Arg-Trp-Cys]-NH-(CH₂)₆-NH₂ |
| 118 | Ac-Tyr-Arg-cyclo[Cys-Glu-His-D-Phe-Arg-Trp-Cys]-Glu-NH₂ |
| 119 | Ac-Tyr-Arg-cyclo[Cys-Glu-His-D-Phe-Arg-Trp-Cys]-Ser-Pro-NH₂ |
| 120 | Ac-Tyr-Arg-cyclo[Cys-Glu-His-D-Phe-Arg-Trp-Cys]-Ser-Pro alcohol |
| 121 | Ac-Tyr-Arg-cyclo[Cys-Glu-His-D-Phe-Arg-Trp-Cys]-Lys-Pro-NH₂ |
| 122 | Ac-Tyr-Arg-cyclo[Cys-Glu-His-D-Phe-Arg-Trp-Cys]-Lys-Pro alcohol |
| 123 | Ac-Tyr-Arg-cyclo[Cys-Glu-His-D-Phe-Arg-Trp-Cys]-Arg-Phe-NH₂ |
| 124 | Ac-Tyr-Cit-cyclo[Cys-Glu-His-D-Phe-Arg-Trp-Cys]-NH₂ |
| 125 | Ac-Tyr-Cit-cyclo[Cys-Glu-(1-Me-His)-D-Phe-Arg-Trp-Cys]-NH₂ |
| 126 | Ac-Tyr-hArg-cyclo[Cys-Glu-His-D-Phe-Arg-Trp-Cys]-NH₂ |
| 127 | Ac-Tyr-(1-β-hArg)-cyclo[Cys-Glu-His-D-Phe-Arg-Trp-Cys]-NH₂ |
| 128 | Ac-Tyr-Lys-cyclo[Cys-Glu-His-D-Phe-Arg-Trp-Cys]-NH₂ |
| 129 | Ac-Tyr-Ser-cyclo[Cys-Glu-His-D-Phe-Arg-Trp-Cys]-NH₂ |
| 130 | Ac-Tyr-Val-cyclo[Cys-Glu-His-D-Phe-Arg-Trp-Cys]-NH₂ |
| 131 | N-succinyl-Tyr-Arg-cyclo[Cys-Glu-His-D-Phe-Arg-Trp-Cys]-OH |
| 132 | cyclo[hCys-His-D-Phe-Arg-Trp-Cys]-NH₂ |
| 133 | cyclo[hCys-His-D-Phe-Arg-Trp-Cys]-OH |
| 134 | cyclo[hCys-His-(4-F-D-Phe)-Arg-Trp-Cys]-NH₂ |
| 135 | cyclo[hCys-His-(4-Cl-D-Phe)-Arg-Trp-Cys]-NH₂ |
| 136 | Ac-cyclo[hCys-His-Phe-Arg-Trp-Cys]-NH₂ |
| 137 | Ac-cyclo[hCys-His-D-Phe-Arg-Trp-Cys]-NH₂ |
| 138 | Ac-cyclo[hCys-His-D-Phe-Arg-Trp-Cys]-OH |
| 139 | Ac-cyclo[hCys-His-(4-F-D-Phe)-Arg-Trp-Cys]-NH₂ |
| 140 | Ac-cyclo[hCys-His-(4-Cl-D-Phe)-Arg-Trp-Cys]-NH₂ |
| 141 | N-cyclopropanecarbonyl-cyclo[hCys-His-D-Phe-Arg-Trp-Cys]-NH₂ |
| 142 | N-cyclobutanecarbonyl-cyclo[hCys-His-D-Phe-Arg-Trp-Cys]-NH₂ |
| 143 | N-cyclopentanecarbonyl-cyclo[hCys-His-D-Phe-Arg-Trp-Cys]-NH₂ |
| 144 | N-cyclohexanecarbonyl-cyclo[hCys-His-D-Phe-Arg-Trp-Cysl-NH₂ |
| 145 | N-hexanoyl-cyclo[hCys-His-D-Phe-Arg-Trp-Cys]-NH₂ |
| 146 | N-benzoyl-cyclo[hCys-His-D-Phe-Arg-Trp-Cys]-NH₂ |
| 147 | 4-phenylbutyryl-cyclo[hCys-His-D-Phe-Arg-Trp-Cys]-NH₂ |
| 148 | 3-guanidinopropionyl-cyclo[hCys-His-D-Phe-Arg-Trp-Cys]-NH₂ |
| 149 | 5-guanidinovaleryl-cyclo[hCys-His-D-Phe-Arg-Trp-Cys]-NH₂ |
| 150 | N-phenylsulfonyl-cyclo[hCys-His-D-Phe-Arg-Trp-Cys]-NH₂ |
| 151 | N-(2-naphthalenesulfonyl)-cyclo[hCys-His-D-Phe-Arg-Trp-Cys]-NH₂ |
| 152 | N-(4-phenylsulfonamido-4-oxo-butyryl)-cyclo[hCys-His-D-Phe-Arg-Trp-Cys]-NH₂ |
| 153 | Arg-cyclo[hCys-His-D-Phe-Arg-Trp-Cys]-NH₂ |
| 154 | D-Arg-cyclo[hCys-His-D-Phe-Arg-Trp-Cys]-NH₂ |
| 155 | Arg-cyclo[hCys-His-D-Phe-Arg-Trp-Cys]-OH |
| 156 | Arg-cyclo[hCys-(1-Me-His)-D-Phe-Arg-Trp-Cys]-NH₂ |
| 157 | Arg-cyclo[hCys-(1-Me-D-His)-D-Phe-Arg-Trp-Cys]-NH₂ |
| 158 | Ac-Arg-cyclo[hCys-His-D-Phe-Arg-Trp-Cys]-NH₂ |
| 159 | Ac-Arg-cyclo[hCys-His-D-Phe-Arg-Trp-Cys]-OH |
| 160 | Ac-nLeu-cyclo[hCys-His-D-Phe-Arg-Trp-Cys]-NH₂ |
| 161 | phenylsulfonyl-Gly-cyclo[hCys-His-D-Phe-Arg-Trp-Cys]-NH₂ |
| 162 | Tyr-Arg-cyclo[hCys-His-D-Phe-Arg-Trp-Cys]-NH₂ |
| 163 | Tyr-Arg-cyclo[hCys-His-D-Phe-Arg-Trp-Cys]-OH |
| 164 | Ac-Tyr-Arg-cyclo[hCys-His-D-Phe-Arg-Trp-Cys]-NH₂ |
| 165 | Ac-Tyr-Arg-cyclo[hCys-His-D-Phe-Arg-Trp-Cys]-OH |
| 166 | Ac-Tyr-Arg-cyclo[hCys-Glu-His-D-Phe-Arg-Trp-Cys]-NH₂ |
| 167 | Ac-cyclo[hCys-His-(β-cyclohexyl-D-Ala)-Arg-Trp-Cys]-NH₂ |
| 168 | Ac-cyclo[hCys-His-D-Phe-Arg-Trp-penicillamine]-NH₂ |
| 169 | Ac-cyclo[hCys-His-(4-Cl-D-Phe)-Arg-Trp-penicillamine]-NH₂ |
| 170 | N-hexanoyl-cyclo[hCys-His-D-Phe-Arg-Trp-penicillamine]-NH₂ |
| 171 | N-cyclopentanecarbonyl-cyclo[hCys-His-D-Phe-Arg-Trp-penicillamine]-NH₂ |
| 172 | N-cyclohexanecarbonyl-cyclo[hCys-His-D-Phe-Arg-Trp-penicillamine]-NH₂ |
| 173 | N-benzoyl-cyclo[hCys-His-D-Phe-Arg-Trp-penicillamine]-NH₂ |
| 174 | 4-phenylbutyryl-cyclo[hCys-His-D-Phe-Arg-Trp-penicillamine]-NH₂ |
| 175 | N-phenylsulfonyl-cyclo[hCys-His-i-Phe-Arg-Trp-penicillamine]-NH₂ |
| 176 | (4-benzenesulfonamide)butyryl-cyclo[hCys-His-D-Phe-Arg-Trp-penicillamine]-NH₂ |
| 177 | Ac-nLeu-cyclo[hCys-His-D-Phe-Arg-Trp-penicillamine]-NH₂ |
| 178 | N-phenylsulfonyl-Gly-cyclo[hCys-His-D-Phe-Arg-Trp-penicillamine]-NH₂ |
| 179 | cyclo[3-thiopropionyl-His-D-Phe-Arg-Trp-hCys]-NH₂ |
| 180 | cyclo[Cys-His-D-Phe-Arg-Trp-hCys]-NH₂ |
| 181 | cyclo[Cys-His-(4-F-D-Phe)-Arg-Trp-hCys]-NH₂ |
| 182 | cyclo[Cys-His-(4-Cl-D-Phe)-Arg-Trp-hCys]-NH₂ |
| 183 | Ac-cyclo[Cys-His-D-Phe-Arg-Trp-hCys]-NH₂ |
| 184 | Ac-cyclo[Cys-His-(4-F-D-Phe)-Arg-Trp-hCys]-NH₂ |
| 185 | Ac-cyclo[Cys-His-(4-Cl-D-Phe)-Arg-Trp-hCys]-NH₂ |
| 186 | Arg-cyclo[Cys-His-D-Phe-Arg-Trp-hCys]-NH₂ |
| 187 | Arg-cyclo[Cys-His-(4-F-D-Phe)-Arg-Trp-hCys]-NH₂ |
| 188 | Arg-cyclo[Cys-His-(4-Cl-D-Phe)-Arg-Trp-hCys]-NH₂ |
| 189 | Ac-Arg-cyclo[Cys-His-D-Phe-Arg-Trp-hCys]-NH₂ |
| 190 | Ac-Arg-cyclo[Cys-His-(4-F-D-Phe)-Arg-Trp-hCys]-NH₂ |
| 191 | Ac-Arg-cyclo[Cys-His-(4-Cl-D-Phe)-Arg-Trp-hCys]-NH₂ |
| 192 | Ac-Tyr-Arg-cyclo[Cys-Glu-His-D-Phe-Arg-Trp-hCys]-NH₂ |
| 193 | Ac-cyclo[hCys-His-D-Phe-Arg-Trp-hCys]-NH₂ |
| 194 | Arg-cyclo[hCys-His-D-Phe-Arg-Trp-hCys]-NH₂ |
| 195 | Ac-Arg-cyclo[hCys-His-D-Phe-Arg-Trp-hCys]-NH₂ |
| 196 | Ac-Tyr-Arg-cyclo[hCys-His-D-Phe-Arg-Trp-hCys]-NH₂ |
| 197 | Ac-Tyr-Arg-cyclo[hCys-Glu-His-D-Phe-Arg-Trp-hCys]-NH₂ |
| 198 | Ac-cyclo(S-CH₂-S)[Cys-His-D-Phe-Arg-Trp-Cys]-NH₂ |

A preferred group for use in the invention includes MC4R agonist peptides having Compound Nos. 48, 52, 132, 137, and 155. More preferred is a group consisting of Compound Numbers 52 and 137. A more preferred compound for use in the present invention is Compound Number 137, denoted by the name Ac-cyclo[hCys-His-D-Phe-Arg-Trp-Cys]-NH₂, as the MC4R agonist peptide. A most preferred compound for use in the present invention is Compound Number 52, denoted by the name AC-D-Arg-cyclo[Cys-Glu-His-D-Phe-Arg-Trp-Cys]-NH₂, as the MC4R agonist peptide.

As used herein, “C₁-C₄ straight or branched alkyl” means a straight chained or branched hydrocarbon having 1 to 4 carbon atoms, which is completely saturated and unsubstituted. “C₃-C₇ cycloalkyl” refers to a saturated, unsubstituted hydrocarbon ring having 3 to 7 carbon atoms. A “C₁-C₄ straight or branched heteroalkyl” refers to a straight chained or branched hydrocarbon having 1 to 4 carbon atoms, which is completely saturated and unsubstituted, that also contains at least one “heteroatom.” A “heteroatom” is nitrogen, oxygen, or sulfur. “C₃-C₇ heterocycloalkyl” refers to a saturated, unsubstituted hydrocarbon ring having 3 to 7 carbon atoms, which also contains at least one “heteroatom.” C₁-C₄ straight or branched alkyl, C₃-C₇ cycloalkyl, C₁-C₄ straight or branched heteroalkyl, and C₃-C₇ heterocycloalkyl may be used as generic modifiers to describe a genus of substituents on another functional group such as a carbonyl, sulfonyl, or sulfonamide. For example, a “C₃-C₇ cycloalkylcarbonyl” refers to a genus of saturated, unsubstituted hydrocarbon rings having 3 to 7 carbon atoms that are bonded to a carbonyl group.

A “C₈-C₁₄ bicyclic aryl” refers to two or three hydrocarbon rings fused together, having 8 to 14 carbon atoms, such as naphthalene. A C₈-C₁₄ bicyclic aryl ring system has at least one aromatic ring. A “5- or 6-membered heteroaryl” refers to a monocyclic aromatic ring having 5 or 6 atoms, of which 1-4 atoms are heteroatoms. An “8- to 14-membered bicyclic heteroaryl” ring refers to two or three hydrocarbon rings fused together, having 8 to 14 atoms, at least one aromatic ring, and 1-4 heteroatoms.

A phenyl, benzyl, benzoyl, C₈-C₁₄ bicyclic aryl, 5- or 6-membered heteroaryl, or 8- to 14-membered bicyclic heteroaryl may be unsubstituted or substituted with C₁-C₄ straight or branched alkyl, F, Cl, Br, —OH, methoxy, phenyl, benzyl, benzoyl, or benzyloxymethyl. Furthermore, phenyl, benzyl, benzoyl, C₈-C₁₄ bicyclic aryl, 5- or 6-membered heteroaryl, and 8- to 14-membered bicyclic heteroaryl may be used as generic modifiers to describe a genus of substituents on another functional group such as a carbonyl, sulfonyl, or sulfonamide. For example, a “C₈-C₁₄ bicyclic arylsulfonyl” refers to a genus of bicyclic aryl rings having 8 to 14 carbon atoms that are bonded to a sulfonyl group.

Modified amino acids are indicated by parentheses around the amino acid and the modification thereto (e.g., (4-Cl-D-Phe) is a 4-chloro modification on the D-isomer of phenylalanine). With respect to moieties depicted in Structural Formula I, Structural Formula II, and Structural Formula III, the single letter designations are as defined and do not refer to single letter amino acids corresponding to those letters.

The letter “D” preceding the above-mentioned 3-letter abbreviations, e.g., “D-Phe,” means the D-form of the amino acid. When the single letter abbreviation is used for an amino acid, a “d” will precede the letter to designate the D-form of the amino acid (e.g., dF=D-Phe).

An “amino alcohol” is an amino acid that has been modified by reducing the carbonyl group of the C-terminus to a methylene group. Amino alcohols are denoted by the general nomenclature “Xaa alcohol,” wherein Xaa is the specific amino acid from which the carbonyl group has been removed. To illustrate, “Ser alcohol” has the structure H₂N—CH(CH₂OH)—CH₂OH as opposed to the Ser amino acid structure of H₂N—CH(CH₂OH)—COOH.

“Single bond,” as used herein, refers to a structure that does not contain an amino acid at the specified position. It is used to signify that an amino acid is absent from that position such that the carbonyl adjacent to that position on one side and the amine adjacent to that position on the other side form a peptide bond with each other.

“*” means that both the D- and L-isomers are possible.

“Ac” refers to acetyl (i.e., —C(O)CH₃).

“Orn” refers to ornithine.

“hCys” refers to homocysteine.

“hArg” refers to homoarginine.

“Lys(ipr)” refers to lysine(N-isopropyl).

“Cit” refers to citrulline.

“nLeu” refers to norleucine.

“Me” refers to methyl.

“OMe” refers to methoxy.

“Cya” refers to cysteic acid.

“Dap” refers to diaminopropionyl.

“Dab” refers to diaminobutyryl.

“Pharmaceutically-acceptable salt” refers to salts of the compounds of the Structural Formula I, Structural Formula II, or Structural Formula III that are substantially non-toxic to mammals. Typical pharmaceutically acceptable salts include those salts prepared by reaction of the compounds of the present invention with a mineral or organic acid or an organic or inorganic base. Such salts are known as acid addition and base addition salts, respectively. It should be recognized that the particular counterion forming a part of any salt of this invention is not of a critical nature, so long as the salt as a whole is pharmaceutically acceptable and as long as the counterion does not contribute undesired qualities to the salt as a whole.

A pharmaceutical “acid addition salt” is a salt formed by reaction of the free base form of a compound of formula I with a pharmaceutical acid, such as described in the Encyclopedia of Pharmaceutical Technology, editors James Swarbrick and James C. Boylan, Vol. 13 (1996), “Preservation of Pharmaceutical Products to Salt Forms of Drugs and Absorption.” Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; l-malate; malonate; d-mandelate; l-mandelate; methanesulfonate; 1,5-napthalenedisulfonate; 2-naphthalenesulfonate; phosphate; salicylate; succinate; sulfate; d-tartrate; l-tartrate; and p-toluenesulfonate.

A pharmaceutical “base addition salt” is a salt formed by reaction of the free acid form of a compound of formula I with a pharmaceutical base, such as described in the Encyclopedia of Pharmaceutical Technology, supra. Specific salt forms include, but are not limited to the: calcium, diethanolamine, diethylamine, ethylenediamine, lysine, magnesium, piperazine, potassium, sodium, and tromethamine (Tris, Trizma) salts.

The term “active ingredient” means the MC4R agonist peptides generically described by Structural Formula I, Structural Formula II, and Structural Formula III, as well as the salts of such compounds.

The term “pharmaceutically acceptable” means that the carrier, diluent, excipients, and salt must be compatible with the other ingredients of the composition and not clinically deleterious to the recipient thereof. Pharmaceutical compositions of the present invention are prepared by procedures known in the art using well-known and readily available ingredients.

The term “agonist” includes any molecule that has affinity for the MC4 receptor, producing a measurable biological activity associated with weight loss in cells, tissues and organisms containing the MC4 receptor. In a similar manner, an “inverse agonist” includes any molecule that has affinity for the MC4 receptor, producing a decreased intrinsic activity of the cell containing the MC4 receptor and is associated with weight gain in cells, tissues, and organisms containing the MC4 receptor. The term “antagonist” includes any molecule that partially or fully blocks, inhibits, or neutralizes a biological activity of the MC4 receptor. Assays measuring such activities are well known in the art.

The term “weight loss” includes any decrease in the mass of a patient. Weight loss may include overall loss of mass by the patient or, alternatively, loss of fat mass by the patient.

The term “obesity,” also called corpulence or fatness, is the excessive accumulation of body fat, usually caused by the consumption of more calories than the body uses. The excess calories are then stored as fat, or adipose tissue. Overweight, if moderate, is not necessarily obesity, particularly in muscular or large-boned individuals. In general, however, a body weight twenty percent or more over the optimum tends to be associated with obesity.

A “subject” or “patient” is a mammal, preferably a human. Nonetheless, other mammals may be subjects or patients, including companion animals such as dogs and cats, laboratory animals such as rats, mice, monkeys, and guinea pigs, and farm animals such as cows, sheep, pigs, and horses.

The term “a patient in need thereof” is a patient either suffering from the claimed pathological condition or sequela thereof or is a patient at a recognized risk thereof as determined by medical diagnosis, i.e., as determined by the attending physician.

The terms “treating,” “treatment,” and “therapy” as used herein refer to the management and care of a patient for the purpose of combating the disease, condition, or disorder. Treating includes the administration of an MC4R agonist peptide to prevent the onset of the symptoms or complications, alleviating the symptoms or complications, or eliminating the disease, condition, or disorder. Treating obesity therefore includes the inhibition of food intake, the inhibition of weight gain, and inducing weight loss in patients in need thereof.

Treatment may include curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

A “therapeutically-effective amount” is the minimal amount of MC4R agonist peptide necessary to induce weight loss. An “effective amount” of the peptide administered to a subject will also depend on the type and severity of the disease or condition and on the characteristics of the subject, such as general health, age, sex, body weight and tolerance to drugs. The recipient patient's physician should determine the therapeutic dose administered in light of the relevant circumstances.

A therapeutically-effective amount can be administered prophylactically to a patient thought to be susceptible to development of a disease or condition. Such amount, when administered prophylactically to a patient, can also be effective to prevent or lessen the severity of the mediated condition. The dosage regimen utilizing the compounds of the present invention is selected by one of ordinary skill in the medical or veterinary arts, in view of a variety of factors, including, without limitation, the route of administration, the prior medical history of the recipient, the pathological condition or symptom being treated, the severity of the condition/symptom being treated, and the age and sex of the recipient patient. However, it will be understood that the therapeutic dose administered will be determined by the attending physician in the light of the relevant circumstances.

Generally, an effective minimum daily dose of a compound of the present invention will exceed about 0.01 mg. Typically, an effective maximum daily dose will not exceed about 1000 mg. More preferably, an effective minimum daily dose will be between about 0.05 mg and 50 mg, more preferably between 0.1 mg and 10 mg. Most preferably, an effective minimum daily dose of an MC4R agonist peptide in the present invention will exceed about 2 μg/kg and will not exceed about 20 μg/kg. The exact dose may be determined, in accordance with the standard practice in the medical arts of “dose titrating” the recipient; that is, initially administering a low dose of the compound, and gradually increasing the does until the desired therapeutic effect is observed. The desired dose may be presented in a single dose or as divided doses administered at appropriate intervals.

The peptides used in the invention may be chemically synthesized. Such methods for synthesis are well known in the art.

### EXAMPLE 1

### Comparison of Continuous Infusion of MC4R Agonist Peptide Versus Daily Subcutaneous Injection

For this type of experiment, two solutions are prepared. First, a 5% dextrose solution is prepared by diluting 5 mL 50% dextrose solution (Biomeda) in 45 mL of sterile water for injection. This dextrose solution is subsequently referred to as “vehicle.” Second, a stock solution of the MC4R agonist peptide (“P1”) to be administered subcutaneously is prepared by dissolving 1.75 mg of P1 in 2 mL of the vehicle. This stock solution is then diluted to 0.088 mg/mL using that vehicle. This solution is subsequently referred to as P1sc solution. Both the P1sc solution and the vehicle are prepared fresh every three days and stored at 4° C. in a sterile capped vial throughout the experiment. A separate solution of MC4R agonist peptide is prepared for continuous infusion using osmotic pumps by dissolving 11.1 mg of P1 in 3 mL of vehicle [3.7 mg/mL]. This solution is subsequently referred to as P1p solution. Ten ALZET® mini-osmotic pumps (implantable infusion pumps that continuously deliver materials to laboratory animals; Model 2002, 14-day payout at 0.5 μL/hour) are loaded using aseptic technique with either 200 μL P1p (n=5) or vehicle (n=5) solution and allowed to prime overnight in sterile 0.9% saline at 37° C. in preparation for implantation into rats.

Twenty rats are selected for this experiment. Ten rats are anaesthetized briefly with isoflorane (3%, Abbott Laboratories). Each anaesthetized rat is implanted with an ALZET® pump using sterile technique. The rats are divided into four groups of five rats: two groups containing pumps and two groups with no pumps. Experimental samples are administered to the rats as follows:

TABLE 1
Administration scheme for a P1 study.

| | | | Approximate |
|---|---|---|---|
| | | | daily dose |
| Group | Substance | Delivery method | (μg/kg active) |
| 1 | MC4-R peptide | Sustained release via pump | 44 |
| | P1 | | |
| 2 | Vehicle | Sustained release via pump | 0 |
| 3 | MC4-R peptide | Daily subcutaneous injection | 44 |
| | P1 | | |
| 4 | Vehicle | Daily subcutaneous injection | 0 |

Each rate is weighed initially, and measurements of body composition are made for each animal using QNMR (quantitative nuclear magnetic resonance). Body mass is measured daily for fourteen days, and the cumulative change in body mass is calculated. Body composition is measured again at the end of the study.

Using a procedure such as that described above, results shown in Tables 2, 3, and 4, below, may be achieved.

TABLE 2
Change in body mass among groups.

Mean change in body mass (g)

| Day | Group 1 | Group 2 | Group 3 | Group 4 |
|---|---|---|---|---|
| 1 | −7.66 | 4.14 | −0.68 | 2.96 |
| 2 | −8.52 | 6.20 | 1.64 | 3.26 |
| 3 | −10.12 | 6.70 | 0.46 | 5.68 |
| 4 | −9.54 | 7.32 | 0.78 | 8.50 |
| 5 | −12.56 | 8.06 | 1.16 | 10.06 |
| 6 | −14.02 | 7.74 | 0.94 | 10.51 |
| 7 | −12.86 | 7.88 | 0.80 | 10.90 |
| 8 | −14.42 | 10.22 | 4.00 | 11.38 |
| 9 | −14.60 | 9.88 | 2.72 | 14.48 |
| 10 | −14.72 | 10.90 | 2.98 | 14.46 |
| 11 | −13.04 | 13.86 | 4.28 | 17.24 |
| 12 | −12.22 | 17.46 | 7.30 | 19.64 |
| 13 | −9.66 | 18.70 | 9.52 | 20.66 |
| 14 | −9.12 | 20.32 | 9.54 | 23.22 |

TABLE 3
Change in fat mass among groups.

Mean fat mass (g)

| Day | Group 1 | Group 2 | Group 3 | Group 4 |
|---|---|---|---|---|
| 0 | 78.171 | 81.725 | 74.252 | 81.312 |
| 14 | 69.273 | 89.887 | 72.912 | 93.182 |
| Change | −8.898 | 8.162 | −1.340 | 11.870 |

TABLE 4
Change in lean mass among groups.

Mean lean mass (g)

| Day | Group 1 | Group 2 | Group 3 | Group 4 |
|---|---|---|---|---|
| 0 | 328.609 | 329.489 | 340.206 | 333.134 |
| 14 | 330.373 | 344.131 | 353.033 | 344.527 |
| Change | 1.764 | 14.642 | 12.827 | 11.393 |

Additionally, the food intake of each animal (mass of food the animal eats in one day) is measured daily during the fourteen-day experiment. Results of this study are shown in Table 5, below.

TABLE 5
Food intake among groups (P1).

Mean daily food intake (g)

| Day | Group 1 | Group 2 | Group 3 | Group 4 |
|---|---|---|---|---|
| 1 | 6.76 | 15.98 | 13.76 | 16.06 |
| 2 | 10.40 | 19.22 | 15.64 | 19.40 |
| 3 | 15.14 | 21.36 | 17.58 | 22.18 |
| 4 | 14.48 | 20.16 | 15.90 | 20.48 |
| 5 | 12.20 | 18.26 | 15.94 | 18.50 |
| 6 | 12.44 | 16.84 | 14.12 | 17.78 |
| 7 | 13.96 | 16.70 | 16.70 | 18.68 |
| 8 | 14.94 | 17.96 | 16.44 | 16.04 |
| 9 | 14.22 | 17.48 | 13.24 | 17.58 |
| 10 | 16.26 | 18.18 | 17.52 | 17.84 |
| 11 | 15.70 | 18.36 | 15.46 | 17.62 |
| 12 | 15.08 | 16.94 | 16.10 | 17.34 |
| 13 | 16.76 | 17.78 | 16.30 | 17.40 |
| 14 | 14.64 | 17.16 | 15.62 | 16.36 |

Continuous subcutaneous infusion of P1 in rats results in improved efficacy over single daily bolus dosing of equivalent P1 [0.044 mg/kg]. Cumulative weight loss in rats infused with P1 is significantly increased over both vehicle treated groups and rats dosed once daily. Decreased fat mass in rats continuously infused with peptide also indicates improved efficacy over daily dosing; however, the change does not reach significance between infused and daily dosed groups.

Experiments such as that described above may be performed on other MC4R agonists and for different time periods. For example, a seven-day study administering another peptide (“P2”) may be performed. A stock solution of the MC4R peptide to be dose subcutaneous is prepared by dissolving 2 mg of P2 in 2 mL of the vehicle. This stock solution is then diluted 0.1 mg/mL using vehicle. This solution is subsequently referred to as P2sc solution. Both the P2sc solution and the vehicle are prepared fresh every three days and stored at 4° C. in a sterile capped vial throughout the experiment. A second solution of the MC4R peptide P2 is prepared by dissolving 5 mg of P2 in 2.4 mL of the vehicle prepared above. This solution is subsequently referred to as P2p solution. Ten ALZET® mini-osmotic pumps (Model 2001, 7-day payout at 1.0 μL/hour) are loaded using aseptic technique with either 200 μL P2 (n=4) or vehicle (n=4) solution and allowed to prime overnight in sterile 0.9% saline at 37° C. in preparation for implantation into rats.

Sixteen rats are selected for this experiment. Ten rats are anaesthetized briefly with isoflorane (prepared above). Each anaesthetized rat is implanted with an ALZET® pump using sterile technique. The rats are divided into four groups of four rats: two groups containing pumps and two groups with no pumps. Experimental samples are administered to the rats as follows:

TABLE 6
Administration scheme for a P2 study.

| | | | Approximate |
|---|---|---|---|
| | | | daily dose |
| Group | Substance | Delivery method | (μg/kg active) |
| 1 | MC4-R peptide | Sustained release via pump | 50 |
| | P2 | | |
| 2 | Vehicle | Sustained release via pump | 0 |
| 3 | MC4-R peptide | Daily subcutaneous injection | 50 |
| | P2 | | |
| 4 | Vehicle | Daily subcutaneous injection | 0 |

Body mass is measured daily for seven days, and the cumulative change in body mass is calculated.

Using a procedure such as that described above, results shown in Table 7, below, may be achieved.

TABLE 7
Change in body mass among groups (P2).

Mean change in body mass (g)

| Day | Group 1 | Group 2 | Group 3 | Group 4 |
|---|---|---|---|---|
| 1 | −5.52 | 7.03 | 5.50 | 5.88 |
| 2 | −3.95 | 8.20 | 9.63 | 7.92 |
| 3 | −10.53 | 1.30 | 5.20 | 4.72 |
| 4 | −8.85 | 3.80 | 5.55 | 6.77 |
| 5 | −11.35 | 2.60 | 7.43 | 8.82 |
| 6 | −11.75 | 4.90 | 8.65 | 11.05 |
| 7 | −13.73 | 6.60 | 9.73 | 12.88 |

Additionally, the food intake of each animal (mass of food the animal eats in one day) is measured daily during the seven-day experiment. Results of this study are shown in Table 8, below.

TABLE 8
Food intake among groups (P2).

Mean daily food intake (g)

| Day | Group 1 | Group 2 | Group 3 | Group 4 |
|---|---|---|---|---|
| 1 | 16.05 | 22.83 | 19.33 | 23.38 |
| 2 | 15.78 | 20.03 | 17.75 | 21.98 |
| 3 | 15.43 | 16.03 | 15.85 | 20.88 |
| 4 | 12.08 | 13.83 | 16.75 | 15.75 |
| 5 | 13.15 | 16.28 | 14.83 | 22.25 |
| 6 | 13.75 | 17.23 | 14.90 | 15.95 |
| 7 | 13.63 | 17.95 | 16.68 | 18.35 |

Continuous subcutaneous infusion of P2 in rats supports P1 study results. Infusion of peptide improved efficacy over single daily bolus dosing of equivalent P2 [0.05 mg/kg]. Cumulative weight loss in rats infused with P2 is significantly increased over both vehicle treated groups and rats dosed once daily.

1. A method of inducing weight loss in a patient, comprising administering by continuous infusion an effective amount of an MC4R agonist peptide to a patient in need thereof, wherein the MC4R agonist peptide is selected from the group consisting of:
Ac-Cya-Arg-cyclo[Cys-Ala-His-D-Phe-Arg-Trp-Cys]-NH₂,
Ac-Tyr-Arg-cyclo[Cys-Ala-His-D-Phe-Arg-Trp-Cys]-NH₂,
Ac-Tyr-Arg-cyclo[Cys-Arg-His-D-Phe-Arg-Trp-Cys]-NH₂,
Ac-Tyr-Arg-cyclo[Cys-Asn-His-D-Phe-Arg-Trp-Cys]-NH₂,
Ac-cyclo[Cys-Asp-His-D-Phe-Arg-Trp-Cys]-NH₂,
Ac-Tyr-Arg-cyclo[Cys-Asp-His-D-Phe-Arg-Trp-Cys]-NH₂,
Ac-cyclo[Cys-Gln-His-D-Phe-Arg-Trp-Cys]-NH₂,
Ac-Tyr-Arg-cyclo[Cys-Gln-His-D-Phe-Arg-Trp-Cys]-OH,
Ac-Tyr-Arg-cyclo[Cys-Gln-His-D-Phe-Arg-Trp-Cys]-OMe,
Tyr-Arg-cyclo[Cys-Gly-His-D-Phe-Arg-Trp-Cys]-NH₂,
Ac-Tyr-Arg-cyclo[Cys-Gly-His-D-Phe-Arg-Trp-Cys]-NH₂,
Ac-Tyr-Arg-cyclo[Cys-His-His-D-Phe-Arg-Trp-Cys]-NH₂,
Ac-Tyr-Arg-cyclo[Cys-Ile-His-D-Phe-Arg-Trp-Cys]-NH₂,
Ac-cyclo[Cys-Leu-His-D-Phe-Arg-Trp-Cys]-NH₂,
Ac-cyclo[Cys-Lys-His-D-Phe-Arg-Trp-Cys]-NH₂,
N-methyl-Tyr-Arg-cyclo[Cys-Met-His-D-Phe-Arg-Trp-Cys]-NH₂,
Ac-Tyr-Arg-cyclo[Cys-Met-His-D-Phe-Arg-Trp-Cys]-NH₂,
Ac-Tyr-Arg-cyclo[Cys-Phe-His-D-Phe-Arg-Trp-Cys]-NH₂,
Ac-Tyr-Arg-cyclo[Cys-Pro-His-D-Phe-Arg-Trp-Cys]-NH₂,
Ac-Tyr-Arg-cyclo[Cys-Ser-His-D-Phe-Arg-Trp-Cys]-NH₂,
Ac-Tyr-Arg-cyclo[Cys-Thr-His-D-Phe-Arg-Trp-Cys]-NH₂,
Ac-Tyr-Arg-cyclo[Cys-Trp-His-D-Phe-Arg-Trp-Cys]-NH₂,
Ac-Tyr-Arg-cyclo[Cys-Tyr-His-D-Phe-Arg-Trp-Cys]-NH₂,
Ac-Tyr-Arg-cyclo[Cys-Val-His-D-Phe-Arg-Trp-Cys]-NH₂,
Ac-Arg-cyclo[Cys-Cya-His-D-Phe-Arg-Trp-Cys]-NH₂,
Ac-D-Arg-cyclo[Cys-Cya-His-D-Phe-Arg-Trp-Cys]-NH₂,
Ac-Tyr-Arg-cyclo[Cys-Cya-His-D-Phe-Arg-Trp-Cys]-NH₂,
cyclo[Cys-Glu-His-D-Phe-Arg-Trp-Cys]-NH₂,
Ac-cyclo[Cys-Glu-His-D-Phe-Arg-Trp-Cys]-NH₂,
Ac-cyclo[Cys-Glu-His-(4-F-D-Phe)-Arg-Trp-Cys]-NH₂,
Ac-cyclo[Cys-Glu-His-(4-Cl-D-Phe)-Arg-Trp-Cys]-NH₂,
Ac-cyclo[Cys-Glu-His-(4-Br-D-Phe)-Arg-Trp-Cys]-NH₂,
Ac-cyclo[Cys-Glu-(1-Me-His)-D-Phe-Arg-Trp-Cys]-NH₂,
Ac-cyclo[Cys-Glu-His-D-Phe-Arg-Trp-Cys]-Lys-Pro-NH₂,
Ac-cyclo[Cys-Glu-His-D-Phe-Arg-Trp-Cys]-Ser-Pro-NH₂,
N-propionyl-cyclo[Cys-Glu-His-D-Phe-Arg-Trp-Cys]-NH₂,
N-butyryl-cyclo[Cys-Glu-His-D-Phe-Arg-Trp-Cys]-NH₂,
N-valeryl-cyclo[Cys-Glu-His-D-Phe-Arg-Trp-Cys]-NH₂,
3-guanidinopropionyl-cyclo[Cys-Glu-His-D-Phe-Arg-Trp-Cys]-NH₂,
4-guanidinobutyryl-cyclo[Cys-Glu-His-D-Phe-Arg-Trp-Cys]-NH₂,
5-guanidinovaleryl-cyclo[Cys-Glu-His-D-Phe-Arg-Trp-Cys]-NH₂,
Ac-diaminopropionyl-cyclo[Cys-Glu-His-D-Phe-Arg-Trp-Cys]-NH₂,
Ac-diaminobutyryl-cyclo[Cys-Glu-His-D-Phe-Arg-Trp-Cys]-NH₂,
Arg-cyclo[Cys-Glu-His-D-Phe-Arg-Trp-Cys]-OH,
D-Arg-cyclo[Cys-Glu-His-D-Phe-Arg-Trp-Cys]-NH₂,
Ac-D-Arg-cyclo[Cys-Glu-His-Phe-Arg-Trp-Cys]-NH₂,
Ac-Arg-cyclo[Cys-Glu-His-D-Phe-Arg-Trp-Cys]-NH₂,
Ac-Arg-cyclo[Cys-Glu-His-D-Phe-Arg-Trp-Cys]-OH,
Ac-Arg-cyclo[Cys-Glu-His-(4-Cl-D-Phe)-Arg-Trp-Cys]-NH₂,
Ac-Arg-cyclo[Cys-Glu-(1-Me-His)-D-Phe-Arg-Trp-Cys]-NH₂,
Ac-D-Arg-cyclo[Cys-Glu-His-D-Phe-Arg-Trp-Cys]-NH₂,
Ac-D-Arg-cyclo[Cys-Glu-His-D-Phe-Arg-Trp-Cys]-OH,
Ac-hArg-cyclo[Cys-Glu-His-D-Phe-Arg-Trp-Cys]-NH₂,
Ac-Cit-cyclo[Cys-Glu-His-D-Phe-Arg-Trp-Cys]-NH₂,
Ac-Cit-cyclo[Cys-Glu-(1-Me-His)-D-Phe-Arg-Trp-Cys]-NH₂,
Ac-Leu-cyclo[Cys-Glu-His-D-Phe-Arg-Trp-Cys]-NH₂,
Ac-Lys-cyclo[Cys-Glu-His-D-Phe-Arg-Trp-Cys]-NH₂,
Ac-Lys(ipr)-cyclo[Cys-Glu-His-D-Phe-Arg-Trp-Cys]-NH₂,
Ac-nLeu-cyclo[Cys-Glu-His-D-Phe-Arg-Trp-Cys]-NH₂,
Ac-nLeu-cyclo[Cys-Glu-His-D-Phe-Arg-Trp-Cys]-Ser-Pro-NH₂,
Ac-Orn-cyclo[Cys-Glu-His-D-Phe-Arg-Trp-Cys]-NH₂,
Ac-Val-cyclo[Cys-Glu-His-D-Phe-Arg-Trp-Cys]-NH₂,
N-(2-naphthalenesulfonyl)-D-Arg-cyclo[Cys-Glu-His-D-Phe-Arg-Trp-Cys]-NH₂,
N-(2-naphthalenesulfonylamino-4-oxo-butyryl)-D-Arg-cyclo[Cys-Glu-His-D-Phe-Arg-Trp-Cys]-NH₂,
3-(4-hydroxyphenyl)propionyl-Arg-cyclo[Cys-Glu-His-D-Phe-Arg-Trp-Cys]-NH₂,
3-(4-methylbenzoyl)propionyl-Arg-cyclo[Cys-Glu-His-D-Phe-Arg-Trp-Cys]-NH₂,
Tyr-Arg-cyclo[Cys-Glu-His-D-Phe-Arg-Trp-Cys]-NH₂,
Tyr-Arg-cyclo[Cys-Glu-His-D-Phe-Arg-Trp-Cys]-OH,
Tyr-Arg-cyclo[Cys-Glu-His-D-Phe-Arg-Trp-Cys]-NH—(CH₂)₆—NH₂,
Tyr-Arg-cyclo[Cys-Glu-His-D-Phe-Arg-Trp-Cys]-Glu-NH₂,
Ac-Tyr-Arg-cyclo[Cys-Glu-His-D-Phe-Arg-Trp-Cys]-NH₂,
Ac-Tyr-Arg-cyclo[Cys-Glu-His-D-Phe-Arg-Trp-Cys]-OH,
N-succinyl-Tyr-Arg-cyclo[Cys-Glu-His-D-Phe-Arg-Trp-Cys]-NH₂,
N-glutaryl-Tyr-Arg-cyclo[Cys-Glu-His-D-Phe-Arg-Trp-Cys]-NH₂,
N-glutaryl-Tyr-Arg-cyclo[Cys-Glu-His-D-Phe-Arg-Trp-Cys]-OH,
gluconoyl-Tyr-Arg-cyclo[Cys-Glu-His-D-Phe-Arg-Trp-Cys]-NH₂,
Ac-Tyr-Arg-cyclo[Cys-Glu-His-D-Phe-Arg-Trp-Cys] alcohol,
Ac-Tyr-D-Arg-cyclo[Cys-Glu-His-D-Phe-Arg-Trp-Cys]-NH₂,
Ac-Tyr-Arg-cyclo[D-Cys-Glu-His-D-Phe-Arg-Trp-Cys]-NH₂,
Ac-Tyr-Arg-cyclo[Cys-Glu-(1-Me-His)-D-Phe-Arg-Trp-Cys]-NH₂,
Ac-Tyr-Arg-cyclo[Cys-Glu-(1-Me-D-His)-D-Phe-Arg-Trp-Cys]-NH₂,
Ac-Tyr-Arg-cyclo[Cys-Glu-His-(4-F-D-Phe)-Arg-Trp-Cys]-NH₂,
Ac-Tyr-Arg-cyclo[Cys-Glu-(1-Me-His)-(4-F-D-Phe)-Arg-Trp-Cys]-NH₂,
Ac-Tyr-Arg-cyclo[Cys-Glu-(1-Me-D-His)-(4-F-D-Phe)-Arg-Trp-Cys]-NH₂,
Ac-Tyr-Arg-cyclo[Cys-Glu-His-(4-Cl-D-Phe)-Arg-Trp-Cys]-NH₂,
Ac-Arg-cyclo[Cys-Glu-(1-Me-His)-(4-Cl-D-Phe)-Arg-Trp-Cys]-NH₂,
Ac-Tyr-Arg-cyclo[Cys-Glu-(1-Me-D-His)-(4-Cl-D-Phe)-Arg-Trp-Cys]-NH₂,
Ac-Tyr-Arg-cyclo[Cys-Glu-His-(4-Br-D-Phe)-Arg-Trp-Cys]-NH₂,
Ac-Tyr-Arg-cyclo[Cys-Glu-(1-Me-His)-(4-Br-D-Phe)-Arg-Trp-Cys]-NH₂,
Ac-Tyr-Arg-cyclo[Cys-Glu-(1-Me-D-His)-(4-Br-D-Phe)-Arg-Trp-Cys]-NH₂,
Ac-Tyr-Arg-cyclo[Cys-Glu-His-(4-Me-D-Phe)-Arg-Trp-Cys]-NH₂,
Ac-Tyr-Arg-cyclo[Cys-Glu-His-(4-OMe-D-Phe)-Arg-Trp-Cys]-NH₂,
Ac-Tyr-Arg-cyclo[Cys-Glu-(1-Me-His)-(4-OMe-D-Phe)-Arg-Trp-Cys]-NH₂,
Ac-Tyr-Arg-cyclo[Cys-Glu-(1-Me-D-His)-(4-OMe-D-Phe)-Arg-Trp-Cys]-NH₂,
Ac-Tyr-Arg-cyclo[Cys-Glu-(3-Me-His)-D-Phe-Arg-Trp-Cys]-NH₂,
Ac-Tyr-Arg-cyclo[Cys-Glu-(5-Me-His)-D-Phe-Arg-Trp-Cys]-NH₂,
Ac-Tyr-Arg-cyclo[Cys-Glu-(5-Me-D-His)-D-Phe-Arg-Trp-Cys]-NH₂,
Ac-Tyr-Arg-cyclo[Cys-Glu-(1-benzyl-His)-D-Phe-Arg-Trp-Cys]-NH₂,
Ac-Tyr-Arg-cyclo[Cys-Glu-(1-benzyl-D-His)-D-Phe-Arg-Trp-Cys]-NH₂,
Ac-Tyr-Arg-cyclo[Cys-Glu-(1-Bom-His)-D-Phe-Arg-Trp-Cys]-NH₂,
Ac-Tyr-Arg-cyclo[Cys-Glu-(1-pyrazolyl-Ala)-D-Phe-Arg-Trp-Cys]-NH₂,
Ac-Tyr-Arg-cyclo[Cys-Glu-(4-phenyl-1H-imidazol-2-yl-Ala)-D-Phe-Arg-Trp-Cys]-NH₂,
Ac-Tyr-Arg-cyclo[Cys-Glu-(4-phenyl-1H-imidazol-2-yl-D-Ala)-D-Phe-Arg-Trp-Cys]-NH₂,
Ac-Tyr-Arg-cyclo[Cys-Glu-(2-pyrazine-Ala)-D-Phe-Arg-Trp-Cys]-NH₂,
Ac-Tyr-Arg-cyclo[Cys-Glu-(β-(1,2,4-triazol-3-yl))-Ala)-D-Phe-Arg-Trp-Cys]-NH₂,
Ac-Tyr-Arg-cyclo[Cys-Glu-(β-(1,2,4-triazol-3-yl))-D-Ala)-D-Phe-Arg-Trp-Cys]-NH₂,
Ac-Tyr-Arg-cyclo[Cys-Glu-(β-((1-benzyl)-1,2,4-triazol-3-yl))-Ala)-D-Phe-Arg-Trp-Cys]-NH₂,
Ac-Tyr-Arg-cyclo[Cys-Glu-(β-((1-benzyl)-1,2,4-triazol-3-yl))-D-Ala)-D-Phe-Arg-Trp-Cys]-NH₂,
Ac-Tyr-Arg-cyclo[Cys-Glu-(β-(2-furyl)-Ala)-D-Phe-Arg-Trp-Cys]-NH₂,
Ac-Tyr-Arg-cyclo[Cys-Glu-(β-(thien-2-yl)-Ala)-D-Phe-Arg-Trp-Cys]-NH₂,
Ac-Tyr-Arg-cyclo[Cys-Glu-(β-(1,3-thiazol-4-yl)-Ala)-D-Phe-Arg-Trp-Cys]-NH₂,
Ac-Tyr-Arg-cyclo[Cys-Glu-(β-(pyridin-4-yl)-Ala)-D-Phe-Arg-Trp-Cys]-NH₂,
Ac-Tyr-Arg-cyclo[Cys-Glu-His-D-Phe-Arg-Trp-Cys]-glycinol,
Ac-Tyr-Arg-cyclo[Cys-Glu-His-D-Phe-Arg-Trp-Cys]-2-(2-aminoethoxy)ethanol,
Ac-Tyr-Arg-cyclo[Cys-Glu-His-D-Phe-Arg-Trp-Cys]-Ser alcohol,
Ac-Tyr-Arg-cyclo[Cys-Glu-His-D-Phe-Arg-Trp-Cys]-NH—(CH₂)₆—NH₂,
Ac-Tyr-Arg-cyclo[Cys-Glu-His-D-Phe-Arg-Trp-Cys]-Glu-NH₂,
Ac-Tyr-Arg-cyclo[Cys-Glu-His-D-Phe-Arg-Trp-Cys]-Ser-pro-NH₂,
Ac-Tyr-Arg-cyclo[Cys-Glu-His-D-Phe-Arg-Trp-Cys]-Ser-Pro alcohol,
Ac-Tyr-Arg-cyclo[Cys-Glu-His-D-Phe-Arg-Trp-Cys]-Lys-Pro-NH₂,
Ac-Tyr-Arg-cyclo[Cys-Glu-His-D-Phe-Arg-Trp-Cys]-Lys-Pro alcohol,
Ac-Tyr-Arg-cyclo[Cys-Glu-His-D-Phe-Arg-Trp-Cys]-Arg-Phe-NH₂,
Ac-Tyr-Cit-cyclo[Cys-Glu-His-D-Phe-Arg-Trp-Cys]-NH₂,
Ac-Tyr-Cit-cyclo[Cys-Glu-(1-Me-His)-D-Phe-Arg-Trp-Cys]-NH₂,
Ac-Tyr-hArg-cyclo[Cys-Glu-His-D-Phe-Arg-Trp-Cys]-NH₂,
Ac-Tyr-(1-β-hArg)-cyclo[Cys-Glu-His-D-Phe-Arg-Trp-Cys]-NH₂,
Ac-Tyr-Lys-cyclo[Cys-Glu-His-D-Phe-Arg-Trp-Cys]-NH₂,
Ac-Tyr-Ser-cyclo[Cys-Glu-His-D-Phe-Arg-Trp-Cys]-NH₂,
Ac-Tyr-Val-cyclo[Cys-Glu-His-D-Phe-Arg-Trp-Cys]-NH₂,
N-succinyl-Tyr-Arg-cyclo[Cys-Glu-His-D-Phe-Arg-Trp-Cys]-OH,
cyclo[hCys-His-D-Phe-Arg-Trp-Cys]-NH₂,
cyclo[hCys-His-D-Phe-Arg-Trp-Cys]-OH,
cyclo[hCys-His-(4-F-D-Phe)-Arg-Trp-Cys]-NH₂,
cyclo[hCys-His-(4-Cl-D-Phe)-Arg-Trp-Cys]-NH₂,
Ac-cyclo[hCys-His-Phe-Arg-Trp-Cys]-NH₂,
Ac-cyclo[hCys-His-D-Phe-Arg-Trp-Cys]-NH₂,
Ac-cyclo[hCys-His-D-Phe-Arg-Trp-Cys]-OH,
Ac-cyclo[hCys-His-(4-F-D-Phe)-Arg-Trp-Cys]-NH₂,
Ac-cyclo[hCys-His-(4-Cl-D-Phe)-Arg-Trp-Cys]-NH₂,
N-cyclopropanecarbonyl-cyclo[hCys-His-D-Phe-Arg-Trp-Cys]-NH₂,
N-cyclobutanecarbonyl-cyclo[hCys-His-D-Phe-Arg-Trp-Cys]-NH₂,
N-cyclopentanecarbonyl-cyclo[hCys-His-D-Phe-Arg-Trp-Cys]-NH₂,
N-cyclohexanecarbonyl-cyclo[hCys-His-D-Phe-Arg-Trp-Cys]-NH₂,
N-hexanoyl-cyclo[hCys-His-D-Phe-Arg-Trp-Cys]-NH₂,
N-benzoyl-cyclo[hCys-His-D-Phe-Arg-Trp-Cys]-NH₂,
4-phenylbutyryl-cyclo[hCys-His-D-Phe-Arg-Trp-Cys]-NH₂,
3-guanidinopropionyl-cyclo[hCys-His-D-Phe-Arg-Trp-Cys]-NH₂,
5-guanidinovaleryl-cyclo[hCys-His-D-Phe-Arg-Trp-Cys]-NH₂,
N-phenylsulfonyl-cyclo[hCys-His-D-Phe-Arg-Trp-Cys]-NH₂,
N-(2-naphthalenesulfonyl)-cyclo[hCys-His-D-Phe-Arg-Trp-Cys]-NH₂,
N-(4-phenylsulfonamido-4-oxo-butyryl)-cyclo[hCys-His-D-Phe-Arg-Trp-Cys]-NH₂,
Arg-cyclo[hCys-His-D-Phe-Arg-Trp-Cys]-NH₂,
D-Arg-cyclo[hCys-His-D-Phe-Arg-Trp-Cys]-NH₂,
Arg-cyclo[hCys-His-D-Phe-Arg-Trp-Cys]-OH,
Arg-cyclo[hCys-(1-Me-His)-D-Phe-Arg-Trp-Cys]-NH₂,
Arg-cyclo[hCys-(1-Me-D-His)-D-Phe-Arg-Trp-Cys]-NH₂,
Ac-Arg-cyclo[hCys-His-D-Phe-Arg-Trp-Cys]-NH₂,
Ac-Arg-cyclo[hCys-His-D-Phe-Arg-Trp-Cys]-OH,
Ac-nLeu-cyclo[hCys-His-D-Phe-Arg-Trp-Cys]-NH₂,
phenylsulfonyl-Gly-cyclo[hCys-His-D-Phe-Arg-Trp-Cys]-NH₂,
Tyr-Arg-cyclo[hCys-His-D-Phe-Arg-Trp-Cys]-NH₂,
Tyr-Arg-cyclo[hCys-His-D-Phe-Arg-Trp-Cys]-OH,
Ac-Tyr-Arg-cyclo[hCys-His-D-Phe-Arg-Trp-Cys]-NH₂,
Ac-Tyr-Arg-cyclo[hCys-His-D-Phe-Arg-Trp-Cys]-OH,
Ac-Tyr-Arg-cyclo[hCys-Glu-His-D-Phe-Arg-Trp-Cys]-NH₂,
Ac-cyclo[hCys-His-(β-cyclohexyl-D-Ala)-Arg-Trp-Cys]-NH₂,
Ac-cyclo[hCys-His-D-Phe-Arg-Trp-penicillamine]-NH₂,
Ac-cyclo[hCys-His-(4-Cl-D-Phe)-Arg-Trp-penicillamine]-NH₂,
N-hexanoyl-cyclo[hCys-His-D-Phe-Arg-Trp-penicillamine]-NH₂,
N-cyclopentanecarbonyl-cyclo[hCys-His-D-Phe-Arg-Trp-penicillamine]-NH₂,
N-cyclohexanecarbonyl-cyclo[hCys-His-D-Phe-Arg-Trp-penicillamine]-NH₂,
N-benzoyl-cyclo[hCys-His-D-Phe-Arg-Trp-penicillamine]-NH₂,
4-phenylbutyryl-cyclo[hCys-His-D-Phe-Arg-Trp-penicillamine]-NH₂,
N-phenylsulfonyl-cyclo[hCys-His-D-Phe-Arg-Trp-penicillamine]-NH₂,
(4-benzenesulfonamide)butyryl-cyclo[hCys-His-D-Phe-Arg-Trp-penicillamine]-NH₂,
Ac-nLeu-cyclo[hCys-His-D-Phe-Arg-Trp-penicillamine]-NH₂,
N-phenylsulfonyl-Gly-cyclo[hCys-His-D-Phe-Arg-Trp-penicillamine]-NH₂,
cyclo[3-thiopropionyl-His-D-Phe-Arg-Trp-hCys]-NH₂,
cyclo[Cys-His-D-Phe-Arg-Trp-hCys]-NH₂,
cyclo[Cys-His-(4-F-D-Phe)-Arg-Trp-hCys]-NH₂,
cyclo[Cys-His-(4-Cl-D-Phe)-Arg-Trp-hCys]-NH₂,
Ac-cyclo[Cys-His-D-Phe-Arg-Trp-hCys]-NH₂,
Ac-cyclo[Cys-His-(4-F-D-Phe)-Arg-Trp-hCys]-NH₂,
Ac-cyclo[Cys-His-(4-Cl-D-Phe)-Arg-Trp-hCys]-NH₂,
Arg-cyclo[Cys-His-D-Phe-Arg-Trp-hCys]-NH₂,
Arg-cyclo[Cys-His-(4-F-D-Phe)-Arg-Trp-hCys]-NH₂,
Arg-cyclo[Cys-His-(4-Cl-D-Phe)-Arg-Trp-hCys]-NH₂,
Ac-Arg-cyclo[Cys-His-D-Phe-Arg-Trp-hCys]-NH₂,
Ac-Arg-cyclo[Cys-His-(4-F-D-Phe)-Arg-Trp-hCys]-NH₂,
Ac-Arg-cyclo[Cys-His-(4-Cl-D-Phe)-Arg-Trp-hCys]-NH₂,
Ac-Tyr-Arg-cyclo[Cys-Glu-His-D-Phe-Arg-Trp-hCys]-NH₂,
Ac-cyclo[hCys-His-D-Phe-Arg-Trp-hCys]-NH₂,
Arg-cyclo[hCys-His-D-Phe-Arg-Trp-hCys]-NH₂,
Ac-Arg-cyclo[hCys-His-D-Phe-Arg-Trp-hCys]-NH₂,
Ac-Tyr-Arg-cyclo[hCys-His-D-Phe-Arg-Trp-hCys]-NH₂,
Ac-Tyr-Arg-cyclo[hCys-Glu-His-D-Phe-Arg-Trp-hCys]-NH₂, and
Ac-cyclo(S—CH₂—S)[Cys-His-D-Phe-Arg-Trp-Cys]-NH₂.

2. A method for treating obesity in a patient, comprising administering by continuous infusion an effective amount of an MC4R agonist peptide to a patient in need thereof, wherein the MC4R agonist peptide is selected from the group consisting of:
Ac-Cya-Arg-cyclo[Cys-Ala-His-D-Phe-Arg-Trp-Cys]-NH₂,
Ac-Tyr-Arg-cyclo[Cys-Ala-His-D-Phe-Arg-Trp-Cys]-NH₂,
Ac-Tyr-Arg-cyclo[Cys-Arg-His-D-Phe-Arg-Trp-Cys]-NH₂,
Ac-Tyr-Arg-cyclo[Cys-Asn-His-D-Phe-Arg-Trp-Cys]-NH₂,
Ac-cyclo[Cys-Asp-His-D-Phe-Arg-Trp-Cys]-NH₂,
Ac-Tyr-Arg-cyclo[Cys-Asp-His-D-Phe-Arg-Trp-Cys]-NH₂,
Ac-cyclo[Cys-Gln-His-D-Phe-Arg-Trp-Cys]-NH₂,
Ac-Tyr-Arg-cyclo[Cys-Gln-His-D-Phe-Arg-Trp-Cys]-OH,
Ac-Tyr-Arg-cyclo[Cys-Gln-His-D-Phe-Arg-Trp-Cys]-OMe,
Tyr-Arg-cyclo[Cys-Gly-His-D-Phe-Arg-Trp-Cys]-NH₂,
Ac-Tyr-Arg-cyclo[Cys-Gly-His-D-Phe-Arg-Trp-Cys]-NH₂,
Ac-Tyr-Arg-cyclo[Cys-His-His-D-Phe-Arg-Trp-Cys]-NH₂,
Ac-Tyr-Arg-cyclo[Cys-Ile-His-D-Phe-Arg-Trp-Cys]-NH₂,
Ac-cyclo[Cys-Leu-His-D-Phe-Arg-Trp-Cys]-NH₂,
Ac-cyclo[Cys-Lys-His-D-Phe-Arg-Trp-Cys]-NH₂,
N-methyl-Tyr-Arg-cyclo[Cys-Met-His-D-Phe-Arg-Trp-Cys]-NH₂,
Ac-Tyr-Arg-cyclo[Cys-Met-His-D-Phe-Arg-Trp-Cys]-NH₂,
Ac-Tyr-Arg-cyclo[Cys-Phe-His-D-Phe-Arg-Trp-Cys]-NH₂,
Ac-Tyr-Arg-cyclo[Cys-Pro-His-D-Phe-Arg-Trp-Cys]-NH₂,
Ac-Tyr-Arg-cyclo[Cys-Ser-His-D-Phe-Arg-Trp-Cys]-NH₂,
Ac-Tyr-Arg-cyclo[Cys-Thr-His-D-Phe-Arg-Trp-Cys]-NH₂,
Ac-Tyr-Arg-cyclo[Cys-Trp-His-D-Phe-Arg-Trp-Cys]-NH₂,
Ac-Tyr-Arg-cyclo[Cys-Tyr-His-D-Phe-Arg-Trp-Cys]-NH₂,
Ac-Tyr-Arg-cyclo[Cys-Val-His-D-Phe-Arg-Trp-Cys]-NH₂,
Ac-Arg-cyclo[Cys-Cya-His-D-Phe-Arg-Trp-Cys]-NH₂,
Ac-D-Arg-cyclo[Cys-Cya-His-D-Phe-Arg-Trp-Cys]-NH₂,
Ac-Tyr-Arg-cyclo[Cys-Cya-His-D-Phe-Arg-Trp-Cys]-NH₂,
cyclo[Cys-Glu-His-D-Phe-Arg-Trp-Cys]-NH₂,
Ac-cyclo[Cys-Glu-His-D-Phe-Arg-Trp-Cys]-NH₂,
Ac-cyclo[Cys-Glu-His-(4-F-D-Phe)-Arg-Trp-Cys]-NH₂,
Ac-cyclo[Cys-Glu-His-(4-Cl-D-Phe)-Arg-Trp-Cys]-NH₂,
Ac-cyclo[Cys-Glu-His-(4-Br-D-Phe)-Arg-Trp-Cys]-NH₂,
Ac-cyclo[Cys-Glu-(1-Me-His)-D-Phe-Arg-Trp-Cys]-NH₂,
Ac-cyclo[Cys-Glu-His-D-Phe-Arg-Trp-Cys]-Lys-Pro-NH₂,
Ac-cyclo[Cys-Glu-His-D-Phe-Arg-Trp-Cys]-Ser-Pro-NH₂,
N-propionyl-cyclo[Cys-Glu-His-D-Phe-Arg-Trp-Cys]-NH₂,
N-butyryl-cyclo[Cys-Glu-His-D-Phe-Arg-Trp-Cys]-NH₂,
N-valeryl-cyclo[Cys-Glu-His-D-Phe-Arg-Trp-Cys]-NH₂,
3-guanidinopropionyl-cyclo[Cys-Glu-His-D-Phe-Arg-Trp-Cys]-NH₂,
4-guanidinobutyryl-cyclo[Cys-Glu-His-D-Phe-Arg-Trp-Cys]-NH₂,
5-guanidinovaleryl-cyclo[Cys-Glu-His-D-Phe-Arg-Trp-Cys]-NH₂,
Ac-diaminopropionyl-cyclo[Cys-Glu-His-D-Phe-Arg-Trp-Cys]-NH₂,
Ac-diaminobutyryl-cyclo[Cys-Glu-His-D-Phe-Arg-Trp-Cys]-NH₂,
Arg-cyclo[Cys-Glu-His-D-Phe-Arg-Trp-Cys]-OH,
D-Arg-cyclo[Cys-Glu-His-D-Phe-Arg-Trp-Cys]-NH₂,
Ac-D-Arg-cyclo[Cys-Glu-His-Phe-Arg-Trp-Cys]-NH₂,
Ac-Arg-cyclo[Cys-Glu-His-D-Phe-Arg-Trp-Cys]-NH₂,
Ac-Arg-cyclo[Cys-Glu-His-D-Phe-Arg-Trp-Cys]-OH,
Ac-Arg-cyclo[Cys-Glu-His-(4-Cl-D-Phe)-Arg-Trp-Cys]-NH₂,
Ac-Arg-cyclo[Cys-Glu-(1-Me-His)-D-Phe-Arg-Trp-Cys]-NH₂,
Ac-D-Arg-cyclo[Cys-Glu-His-D-Phe-Arg-Trp-Cys]-NH₂,
Ac-D-Arg-cyclo[Cys-Glu-His-D-Phe-Arg-Trp-Cys]-OH,
Ac-hArg-cyclo[Cys-Glu-His-D-Phe-Arg-Trp-Cys]-NH₂,
Ac-Cit-cyclo[Cys-Glu-His-D-Phe-Arg-Trp-Cys]-NH₂,
Ac-Cit-cyclo[Cys-Glu-(1-Me-His)-D-Phe-Arg-Trp-Cys]-NH₂,
Ac-Leu-cyclo[Cys-Glu-His-D-Phe-Arg-Trp-Cys]-NH₂,
Ac-Lys-cyclo[Cys-Glu-His-D-Phe-Arg-Trp-Cys]-NH₂,
Ac-Lys(ipr)-cyclo[Cys-Glu-His-D-Phe-Arg-Trp-Cys]-NH₂,
Ac-nLeu-cyclo[Cys-Glu-His-D-Phe-Arg-Trp-Cys]-NH₂,
Ac-nLeu-cyclo[Cys-Glu-His-D-Phe-Arg-Trp-Cys]-Ser-Pro-NH₂,
Ac-Orn-cyclo[Cys-Glu-His-D-Phe-Arg-Trp-Cys]-NH₂,
Ac-Val-cyclo[Cys-Glu-His-D-Phe-Arg-Trp-Cys]-NH₂,
N-(2-naphthalenesulfonyl)-D-Arg-cyclo[Cys-Glu-His-D-Phe-Arg-Trp-Cys]-NH₂,
N-(2-naphthalenesulfonylamino-4-oxo-butyryl)-D-Arg-cyclo[Cys-Glu-His-D-Phe-Arg-Trp-Cys]-NH₂,
3-(4-hydroxyphenyl)propionyl-Arg-cyclo[Cys-Glu-His-D-Phe-Arg-Trp-Cys]-NH₂,
3-(4-methylbenzoyl)propionyl-Arg-cyclo[Cys-Glu-His-D-Phe-Arg-Trp-Cys]-NH₂,
Tyr-Arg-cyclo[Cys-Glu-His-D-Phe-Arg-Trp-Cys]-NH₂,
Tyr-Arg-cyclo[Cys-Glu-His-D-Phe-Arg-Trp-Cys]-OH,
Tyr-Arg-cyclo[Cys-Glu-His-D-Phe-Arg-Trp-Cys]-NH—(CH₂)₆—NH₂,
Tyr-Arg-cyclo[Cys-Glu-His-D-Phe-Arg-Trp-Cys]-Glu-NH₂,
Ac-Tyr-Arg-cyclo[Cys-Glu-His-D-Phe-Arg-Trp-Cys]-NH₂,
Ac-Tyr-Arg-cyclo[Cys-Glu-His-D-Phe-Arg-Trp-Cys]-OH,
N-succinyl-Tyr-Arg-cyclo[Cys-Glu-His-D-Phe-Arg-Trp-Cys]-NH₂,
N-glutaryl-Tyr-Arg-cyclo[Cys-Glu-His-D-Phe-Arg-Trp-Cys]-NH₂,
N-glutaryl-Tyr-Arg-cyclo[Cys-Glu-His-D-Phe-Arg-Trp-Cys]-OH,
gluconoyl-Tyr-Arg-cyclo[Cys-Glu-His-D-Phe-Arg-Trp-Cys]-NH₂,
Ac-Tyr-Arg-cyclo[Cys-Glu-His-D-Phe-Arg-Trp-Cys] alcohol,
Ac-Tyr-D-Arg-cyclo[Cys-Glu-His-D-Phe-Arg-Trp-Cys]-NH₂,
Ac-Tyr-Arg-cyclo[D-Cys-Glu-His-D-Phe-Arg-Trp-Cys]-NH₂,
Ac-Tyr-Arg-cyclo[Cys-Glu-(1-Me-His)-D-Phe-Arg-Trp-Cys]-NH₂,
Ac-Tyr-Arg-cyclo[Cys-Glu-(1-Me-D-His)-D-Phe-Arg-Trp-Cys]-NH₂,
Ac-Tyr-Arg-cyclo[Cys-Glu-His-(4-F-D-Phe)-Arg-Trp-Cys]-NH₂,
Ac-Tyr-Arg-cyclo[Cys-Glu-(1-Me-His)-(4-F-D-Phe)-Arg-Trp-Cys]-NH₂,
Ac-Tyr-Arg-cyclo[Cys-Glu-(1-Me-D-His)-(4-F-D-Phe)-Arg-Trp-Cys]-NH₂,
Ac-Tyr-Arg-cyclo[Cys-Glu-His-(4-Cl-D-Phe)-Arg-Trp-Cys]-NH₂,
Ac-Arg-cyclo[Cys-Glu-(1-Me-His)-(4-Cl-D-Phe)-Arg-Trp-Cys]-NH₂,
Ac-Tyr-Arg-cyclo[Cys-Glu-(1-Me-D-His)-(4-Cl-D-Phe)-Arg-Trp-Cys]-NH₂,
Ac-Tyr-Arg-cyclo[Cys-Glu-His-(4-Br-D-Phe)-Arg-Trp-Cys]-NH₂,
Ac-Tyr-Arg-cyclo[Cys-Glu-(1-Me-His)-(4-Br-D-Phe)-Arg-Trp-Cys]-NH₂,
Ac-Tyr-Arg-cyclo[Cys-Glu-(1-Me-D-His)-(4-Br-D-Phe)-Arg-Trp-Cys]-NH₂,
Ac-Tyr-Arg-cyclo[Cys-Glu-His-(4-Me-D-Phe)-Arg-Trp-Cys]-NH₂,
Ac-Tyr-Arg-cyclo[Cys-Glu-His-(4-OMe-D-Phe)-Arg-Trp-Cys]-NH₂,
Ac-Tyr-Arg-cyclo[Cys-Glu-(1-Me-His)-(4-OMe-D-Phe)-Arg-Trp-Cys]-NH₂,
Ac-Tyr-Arg-cyclo[Cys-Glu-(1-Me-D-His)-(4-OMe-D-Phe)-Arg-Trp-Cys]-NH₂,
Ac-Tyr-Arg-cyclo[Cys-Glu-(3-Me-His)-D-Phe-Arg-Trp-Cys]-NH₂,
Ac-Tyr-Arg-cyclo[Cys-Glu-(5-Me-His)-D-Phe-Arg-Trp-Cys]-NH₂,
Ac-Tyr-Arg-cyclo[Cys-Glu-(5-Me-D-His)-D-Phe-Arg-Trp-Cys]-NH₂,
Ac-Tyr-Arg-cyclo[Cys-Glu-(1-benzyl-His)-D-Phe-Arg-Trp-Cys]-NH₂,
Ac-Tyr-Arg-cyclo[Cys-Glu-(1-benzyl-D-His)-D-Phe-Arg-Trp-Cys]-NH₂,
Ac-Tyr-Arg-cyclo[Cys-Glu-(1-Bom-His)-D-Phe-Arg-Trp-Cys]-NH₂,
Ac-Tyr-Arg-cyclo[Cys-Glu-(1-pyrazolyl-Ala)-D-Phe-Arg-Trp-Cys]-NH₂,
Ac-Tyr-Arg-cyclo[Cys-Glu-(4-phenyl-1H-imidazol-2-yl-Ala)-D-Phe-Arg-Trp-Cys]-NH₂,
Ac-Tyr-Arg-cyclo[Cys-Glu-(4-phenyl-1H-imidazol-2-yl-D-Ala)-D-Phe-Arg-Trp-Cys]-NH₂,
Ac-Tyr-Arg-cyclo[Cys-Glu-(2-pyrazine-Ala)-D-Phe-Arg-Trp-Cys]-NH₂,
Ac-Tyr-Arg-cyclo[Cys-Glu-(β-(1,2,4-triazol-3-yl))-Ala)-D-Phe-Arg-Trp-Cys]-NH₂,
Ac-Tyr-Arg-cyclo[Cys-Glu-(β-(1,2,4-triazol-3-yl))-D-Ala)-D-Phe-Arg-Trp-Cys]-NH₂,
Ac-Tyr-Arg-cyclo[Cys-Glu-(β-((1-benzyl)-1,2,4-triazol-3-yl))-Ala)-D-Phe-Arg-Trp-Cys]-NH₂,
Ac-Tyr-Arg-cyclo[Cys-Glu-(β-((1-benzyl)-1,2,4-triazol-3-yl))-D-Ala)-D-Phe-Arg-Trp-Cys]-NH₂,
Ac-Tyr-Arg-cyclo[Cys-Glu-(β-(2-furyl)-Ala)-D-Phe-Arg-Trp-Cys]-NH₂,
Ac-Tyr-Arg-cyclo[Cys-Glu-(β-(thien-2-yl)-Ala)-D-Phe-Arg-Trp-Cys]-NH₂,
Ac-Tyr-Arg-cyclo[Cys-Glu-(β-(1,3-thiazol-4-yl)-Ala)-D-Phe-Arg-Trp-Cys]-NH₂,
Ac-Tyr-Arg-cyclo[Cys-Glu-(β-(pyridin-4-yl)-Ala)-D-Phe-Arg-Trp-Cys]-NH₂,
Ac-Tyr-Arg-cyclo[Cys-Glu-His-D-Phe-Arg-Trp-Cys]-glycinol,
Ac-Tyr-Arg-cyclo[Cys-Glu-His-D-Phe-Arg-Trp-Cys]-2-(2-aminoethoxy)ethanol,
Ac-Tyr-Arg-cyclo[Cys-Glu-His-D-Phe-Arg-Trp-Cys]-Ser alcohol,
Ac-Tyr-Arg-cyclo[Cys-Glu-His-D-Phe-Arg-Trp-Cys]-NH—(CH₂)₆—NH₂,
Ac-Tyr-Arg-cyclo[Cys-Glu-His-D-Phe-Arg-Trp-Cys]-Glu-NH₂,
Ac-Tyr-Arg-cyclo[Cys-Glu-His-D-Phe-Arg-Trp-Cys]-Ser-Pro-NH₂,
Ac-Tyr-Arg-cyclo[Cys-Glu-His-D-Phe-Arg-Trp-Cys]-Ser-Pro alcohol,
Ac-Tyr-Arg-cyclo[Cys-Glu-His-D-Phe-Arg-Trp-Cys]-Lys-Pro-NH₂,
Ac-Tyr-Arg-cyclo[Cys-Glu-His-D-Phe-Arg-Trp-Cys]-Lys-Pro alcohol,
Ac-Tyr-Arg-cyclo[Cys-Glu-His-D-Phe-Arg-Trp-Cys]-Arg-Phe-NH₂,
Ac-Tyr-Cit-cyclo[Cys-Glu-His-D-Phe-Arg-Trp-Cys]-NH₂,
Ac-Tyr-Cit-cyclo[Cys-Glu-(1-Me-His)-D-Phe-Arg-Trp-Cys]-NH₂,
Ac-Tyr-hArg-cyclo[Cys-Glu-His-D-Phe-Arg-Trp-Cys]-NH₂,
Ac-Tyr-(1-β-hArg)-cyclo[Cys-Glu-His-D-Phe-Arg-Trp-Cys]-NH₂,
Ac-Tyr-Lys-cyclo[Cys-Glu-His-D-Phe-Arg-Trp-Cys]-NH₂,
Ac-Tyr-Ser-cyclo[Cys-Glu-His-D-Phe-Arg-Trp-Cys]-NH₂,
Ac-Tyr-Val-cyclo[Cys-Glu-His-D-Phe-Arg-Trp-Cys]-NH₂,
N-succinyl-Tyr-Arg-cyclo[Cys-Glu-His-D-Phe-Arg-Trp-Cys]-OH,
cyclo[hCys-His-D-Phe-Arg-Trp-Cys]-NH₂,
cyclo[hCys-His-D-Phe-Arg-Trp-Cys]-OH,
cyclo[hCys-His-(4-F-D-Phe)-Arg-Trp-Cys]-NH₂,
cyclo[hCys-His-(4-Cl-D-Phe)-Arg-Trp-Cys]-NH₂,
Ac-cyclo[hCys-His-Phe-Arg-Trp-Cys]-NH₂,
Ac-cyclo[hCys-His-D-Phe-Arg-Trp-Cys]-NH₂,
Ac-cyclo[hCys-His-D-Phe-Arg-Trp-Cys]-OH,
Ac-cyclo[hCys-His-(4-F-D-Phe)-Arg-Trp-Cys]-NH₂,
Ac-cyclo[hCys-His-(4-Cl-D-Phe)-Arg-Trp-Cys]-NH₂,
N-cyclopropanecarbonyl-cyclo[hCys-His-D-Phe-Arg-Trp-Cys]-NH₂,
N-cyclobutanecarbonyl-cyclo[hCys-His-D-Phe-Arg-Trp-Cys]-NH₂,
N-cyclopentanecarbonyl-cyclo[hCys-His-D-Phe-Arg-Trp-Cys]-NH₂,
N-cyclohexanecarbonyl-cyclo[hCys-His-D-Phe-Arg-Trp-Cys]-NH₂,
N-hexanoyl-cyclo[hCys-His-D-Phe-Arg-Trp-Cys]-NH₂,
N-benzoyl-cyclo[hCys-His-D-Phe-Arg-Trp-Cys]-NH₂,
4-phenylbutyryl-cyclo[hCys-His-D-Phe-Arg-Trp-Cys]-NH₂,
3-guanidinopropionyl-cyclo[hCys-His-D-Phe-Arg-Trp-Cys]-NH₂,
5-guanidinovaleryl-cyclo[hCys-His-D-Phe-Arg-Trp-Cys]-NH₂,
N-phenylsulfonyl-cyclo[hCys-His-D-Phe-Arg-Trp-Cys]-NH₂,
N-(2-naphthalenesulfonyl)-cyclo[hCys-His-D-Phe-Arg-Try-Cys]-NH₂,
N-(4-phenylsulfonamido-4-oxo-butyryl)-cyclo[hCys-His-D-Phe-Arg-Trp-Cys]-NH₂,
Arg-cyclo[hCys-His-D-Phe-Arg-Trp-Cys]-NH₂,
D-Arg-cyclo[hCys-His-D-Phe-Arg-Trp-Cys]-NH₂,
Arg-cyclo[hCys-His-D-Phe-Arg-Trp-Cys]-OH,
Arg-cyclo[hCys-(1-Me-His)-D-Phe-Arg-Trp-Cys]-NH₂,
Arg-cyclo[hCys-(1-Me-D-His)-D-Phe-Arg-Trp-Cys]-NH₂,
Ac-Arg-cyclo[hCys-His-D-Phe-Arg-Trp-Cys]-NH₂,
Ac-Arg-cyclo[hCys-His-D-Phe-Arg-Trp-Cys]-OH,
Ac-nLeu-cyclo[hCys-His-D-Phe-Arg-Trp-Cys]-NH₂,
phenylsulfonyl-Gly-cyclo[hCys-His-D-Phe-Arg-Trp-Cys]-NH₂,
Tyr-Arg-cyclo[hCys-His-D-Phe-Arg-Trp-Cys]-NH₂,
Tyr-Arg-cyclo[hCys-His-D-Phe-Arg-Trp-Cys]-OH,
Ac-Tyr-Arg-cyclo[hCys-His-D-Phe-Arg-Trp-Cys]-NH₂,
Ac-Tyr-Arg-cyclo[hCys-His-D-Phe-Arg-Trp-Cys]-OH,
Ac-Tyr-Arg-cyclo[hCys-Glu-His-D-Phe-Arg-Trp-Cys]-NH₂,
Ac-cyclo[hCys-His-(β-cyclohexyl-D-Ala)-Arg-Trp-Cys]-NH₂,
Ac-cyclo[hCys-His-D-Phe-Arg-Trp-penicillamine]-NH₂,
Ac-cyclo[hCys-His-(4-Cl-D-Phe)-Arg-Trp-penicillamine]-NH₂,
N-hexanoyl-cyclo[hCys-His-D-Phe-Arg-Trp-penicillamine]-NH₂,
N-cyclopentanecarbonyl-cyclo[hCys-His-D-Phe-Arg-Trp-penicillamine]-NH₂,
N-cyclohexanecarbonyl-cyclo[hCys-His-D-Phe-Arg-Trp-penicillamine]-NH₂,
N-benzoyl-cyclo[hCys-His-D-Phe-Arg-Trp-penicillamine]-NH₂,
4-phenylbutyryl-cyclo[hCys-His-D-Phe-Arg-Trp-penicillamine]-NH₂,
N-phenylsulfonyl-cyclo[hCys-His-D-Phe-Arg-Trp-penicillamine]-NH₂,
(4-benzenesulfonamide)butyryl-cyclo[Cys-His-D-Phe-Arg-Trp-penicillamine]-NH₂,
Ac-nLeu-cyclo[hCys-His-D-Phe-Arg-Trp-penicillamine]-NH₂,
N-phenylsulfonyl-Gly-cyclo[hCys-His-D-Phe-Arg-Trp-penicillamine]-NH₂,
cyclo[3-thiopropionyl-His-D-Phe-Arg-Trp-hCys]-NH₂,
cyclo[Cys-His-D-Phe-Arg-Trp-hCys]-NH₂,
cyclo[Cys-His-(4-F-D-Phe)-Arg-Trp-hCys]-NH₂,
cyclo[Cys-His-(4-Cl-D-Phe)-Arg-Trp-hCys]-NH₂,
Ac-cyclo[Cys-His-D-Phe-Arg-Trp-hCys]-NH₂,
Ac-cyclo[Cys-His-(4-F-D-Phe)-Arg-Trp-hCys]-NH₂,
Ac-cyclo[Cys-His-(4-Cl-D-Phe)-Arg-Trp-hCys]-NH₂,
Arg-cyclo[Cys-His-D-Phe-Arg-Trp-hCys]-NH₂,
Arg-cyclo[Cys-His-(4-F-D-Phe)-Arg-Trp-hCys]-NH₂,
Arg-cyclo[Cys-His-(4-Cl-D-Phe)-Arg-Trp-hCys]-NH₂,
Ac-Arg-cyclo[Cys-His-D-Phe-Arg-Trp-hCys]-NH₂,
Ac-Arg-cyclo[Cys-His-(4-F-D-Phe)-Arg-Trp-hCys]-NH₂,
Ac-Arg-cyclo[Cys-His-(4-Cl-D-Phe)-Arg-Trp-hCys]-NH₂,
Ac-Tyr-Arg-cyclo[Cys-Glu-His-D-Phe-Arg-Trp-hCys]-NH₂,
Ac-cyclo[hCys-His-D-Phe-Arg-Trp-hCys]-NH₂,
Arg-cyclo[hCys-His-D-Phe-Arg-Trp-hCys]-NH₂,
Ac-Arg-cyclo[hCys-His-D-Phe-Arg-Trp-hCys]-NH₂,
Ac-Tyr-Arg-cyclo[hCys-His-D-Phe-Arg-Trp-hCys]-NH₂,
Ac-Tyr-Arg-cyclo[Cys-Glu-His-D-Phe-Arg-Trp-hCys]-NH₂, and
Ac-cyclo(S—CH₂—S)[Cys-His-D-Phe-Arg-Trp-Cys]-NH₂.

3. The method of claim 1, wherein the MC4R agonist peptide is administered using a pump.

4. The method of claim 1, wherein the MC4R agonist peptide is administered using a depot.

5. (canceled)

6. (canceled)

7. (canceled)

8. The method of claim 1, wherein the MC4R agonist peptide is
cyclo[hCys-His-D-Phe-Arg-Trp-Cys]-NH₂,
Ac-cyclo[hCys-His-D-Phe-Arg-Trp-Cys]-NH₂,
Arg-cyclo[hCys-His-D-Phe-Arg-Trp-Cys]-OH,
Ac-Arg-cyclo[Cys-Glu-His-D-Phe-Arg-Trp-Cys]-NH₂, or
Ac-D-Arg-cyclo[Cys-Glu-His-D-Phe-Arg-Trp-Cys]-NH₂.

9. The method of claim 1, wherein the MC4R agonist peptide is Ac-D-Arg-cyclo[Cys-Glu-His-D-Phe-Arg-Trp-Cys]-NH₂.

10. (canceled)

11. (canceled)

12. (canceled)

13. (canceled)

14. (canceled)

15. (canceled)

16. (canceled)

17. (canceled)

18. The method of claim 2, wherein the MC4R agonist peptide is administered using a pump.

19. The method of claim 2, wherein the MC4R agonist peptide is administered using a depot.

20. The method of claim 2, wherein the MC4R agonist peptide is
cyclo[hCys-His-D-Phe-Arg-Trp-Cys]-NH₂,
Ac-cyclo[hCys-His-D-Phe-Arg-Trp-Cys]-NH₂,
Arg-cyclo[hCys-His-D-Phe-Arg-Trp-Cys]-OH,
Ac-Arg-cyclo[Cys-Glu-His-D-Phe-Arg-Trp-Cys]-NH₂, or
Ac-D-Arg-cyclo[Cys-Glu-His-D-Phe-Arg-Trp-Cys]-NH₂.

21. The method of claim 2, wherein the MC4R agonist peptide is Ac-D-Arg-cyclo[Cys-Glu-His-D-Phe-Arg-Trp-Cys]-NH₂.


SEQUENCE LISTING (201 sequences)

SEQ ID NO: 1 - protein, 6 aa, Artificial
  Synthetic construct
  DISULFID (1)..(6)
  MOD_RES (1)..(1): ACETYLATION
  MOD_RES (3)..(3): D form
  MOD_RES (6)..(6): AMIDATION
CHFRWC

SEQ ID NO: 2 - protein, 9 aa, Artificial
  Synthetic construct
  MISC_FEATURE (1)..(1): Xaa = Cysteic acid
  MOD_RES (1)..(1): ACETYLATION
  DISULFID (3)..(9)
  MOD_RES (6)..(6): D form
  MOD_RES (9)..(9): AMIDATION
XRCAHFRWC

SEQ ID NO: 3 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCAHFRWC

SEQ ID NO: 4 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCRHFRWC

SEQ ID NO: 5 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCNHFRWC

SEQ ID NO: 6 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CDHFRWC

SEQ ID NO: 7 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCDHFRWC

SEQ ID NO: 8 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CQHFRWC

SEQ ID NO: 9 - protein, 9 aa, Artificial
  Synthetic construct
  MOD_RES (1)..(1): ACETYLATION
  DISULFID (3)..(9)
  MOD_RES (6)..(6): D form
YRCQHFRWC

SEQ ID NO: 10 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): Methoxy substituted for OH
YRCQHFRWC

SEQ ID NO: 11 - protein, 9 aa, Artificial
  Synthetic construct
  DISULFID (3)..(9)
  MOD_RES (6)..(6): D form
  MOD_RES (9)..(9): AMIDATION
YRCGHFRWC

SEQ ID NO: 12 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCGHFRWC

SEQ ID NO: 13 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCHHFRWC

SEQ ID NO: 14 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCIHFRWC

SEQ ID NO: 15 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CLHFRWC

SEQ ID NO: 16 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CKHFRWC

SEQ ID NO: 17 - protein, 9 aa, Artificial
  Synthetic construct
  MOD_RES (1)..(1): METHYLATION
  DISULFID (3)..(9)
  MOD_RES (6)..(6): D form
  MOD_RES (9)..(9): AMIDATION
YRCMHFRWC

SEQ ID NO: 18 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCMHFRWC

SEQ ID NO: 19 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCFHFRWC

SEQ ID NO: 20 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCPHFRWC

SEQ ID NO: 21 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCSHFRWC

SEQ ID NO: 22 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCTHFRWC

SEQ ID NO: 23 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCWHFRWC

SEQ ID NO: 24 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCYHFRWC

SEQ ID NO: 25 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCVHFRWC

SEQ ID NO: 26 - protein, 8 aa, Artificial
  Synthetic construct
  MOD_RES (1)..(1): ACETYLATION
  DISULFID (2)..(8)
  MISC_FEATURE (3)..(3): Xaa = cysteic acid
  MOD_RES (5)..(5): D form
  MOD_RES (8)..(8): AMIDATION
RCXHFRWC

SEQ ID NO: 27 - protein, 8 aa, Artificial
  Synthetic construct
  MOD_RES (1)..(1): ACETYLATION
  MOD_RES (1)..(1): D form
  DISULFID (2)..(8)
  MISC_FEATURE (3)..(3): Xaa = cysteic acid
  MOD_RES (5)..(5): D form
  MOD_RES (8)..(8): AMIDATION
RCXHFRWC

SEQ ID NO: 28 - protein, 9 aa, Artificial
  Synthetic construct
  MOD_RES (1)..(1): ACETYLATION
  DISULFID (3)..(9)
  MISC_FEATURE (4)..(4): Xaa = cysteic acid
  MOD_RES (6)..(6): D form
  MOD_RES (9)..(9): AMIDATION
YRCXHFRWC

SEQ ID NO: 29 - protein, 7 aa, Artificial
  Synthetic construct
  DISULFID (1)..(7)
  MOD_RES (4)..(4): D form
  MOD_RES (7)..(7): AMIDATION
CEHFRWC

SEQ ID NO: 30 - protein, 7 aa, Artificial
  Synthetic construct
  MOD_RES (1)..(1): ACETYLATION
  DISULFID (1)..(7)
  MOD_RES (4)..(4): D form
  MOD_RES (7)..(7): AMIDATION
CEHFRWC

SEQ ID NO: 31 - protein, 7 aa, Artificial
  Synthetic construct
  MOD_RES (1)..(1): ACETYLATION
  DISULFID (1)..(7)
  MOD_RES (4)..(4): 4-fluoro substituted, D form
  MOD_RES (7)..(7): AMIDATION
CEHFRWC

SEQ ID NO: 32 - protein, 7 aa, Artificial
  Synthetic construct
  MOD_RES (1)..(1): ACETYLATION
  DISULFID (1)..(7)
  MOD_RES (4)..(4): 4-chloro substituted, D form
  MOD_RES (7)..(7): AMIDATION
CEHFRWC

SEQ ID NO: 33 - protein, 7 aa, Artificial
  Synthetic construct
  MOD_RES (1)..(1): ACETYLATION
  DISULFID (1)..(7)
  MOD_RES (4)..(4): 4-bromo substituted, D form
  MOD_RES (7)..(7): AMIDATION
CEHFRWC

SEQ ID NO: 34 - protein, 7 aa, Artificial
  Synthetic construct
  MOD_RES (1)..(1): ACETYLATION
  DISULFID (1)..(7)
  MOD_RES (3)..(3): 1-methyl substituted
  MOD_RES (4)..(4): D form
  MOD_RES (7)..(7): AMIDATION
CEHFRWC

SEQ ID NO: 35 - protein, 9 aa, Artificial
  Synthetic construct
  MOD_RES (1)..(1): ACETYLATION
  DISULFID (1)..(7)
  MOD_RES (4)..(4): D form
  MOD_RES (9)..(9): AMIDATION
CEHFRWCKP

SEQ ID NO: 36 - protein, 9 aa, Artificial
  Synthetic construct
  MOD_RES (1)..(1): ACETYLATION
  DISULFID (1)..(7)
  MOD_RES (4)..(4): D form
  MOD_RES (9)..(9): AMIDATION
CEHFRWCSP

SEQ ID NO: 37 - protein, 7 aa, Artificial
  Synthetic construct
  MOD_RES (1)..(1): N-propionyl substituted
  DISULFID (1)..(7)
  MOD_RES (4)..(4): D form
  MOD_RES (7)..(7): AMIDATION
CEHFRWC

SEQ ID NO: 38 - protein, 7 aa, Artificial
  Synthetic construct
  MOD_RES (1)..(1): N-butyryl substituted
  DISULFID (1)..(7)
  MOD_RES (4)..(4): D form
  MOD_RES (7)..(7): AMIDATION
CEHFRWC

SEQ ID NO: 39 - protein, 7 aa, Artificial
  Synthetic construct
  MOD_RES (1)..(1): N-valeryl substituted
  DISULFID (1)..(7)
  MOD_RES (4)..(4): D form
  MOD_RES (7)..(7): AMIDATION
CEHFRWC

SEQ ID NO: 40 - protein, 7 aa, Artificial
  Synthetic construct
  MOD_RES (1)..(1): 3-guanidinopropionyl substituted
  DISULFID (1)..(7)
  MOD_RES (4)..(4): D form
  MOD_RES (7)..(7): AMIDATION
CEHFRWC

SEQ ID NO: 41 - protein, 7 aa, Artificial
  Synthetic construct
  MOD_RES (1)..(1): 4-guanidinobutyryl substituted
  DISULFID (1)..(7)
  MOD_RES (4)..(4): D form
  MOD_RES (7)..(7): AMIDATION
CEHFRWC

SEQ ID NO: 42 - protein, 7 aa, Artificial
  Synthetic construct
  MOD_RES (1)..(1): 5-guanidinovaleryl substituted
  DISULFID (1)..(7)
  MOD_RES (4)..(4): D form
  MOD_RES (7)..(7): AMIDATION
CEHFRWC

SEQ ID NO: 43 - protein, 7 aa, Artificial
  Synthetic construct
  MOD_RES (1)..(1): acetyl-diaminopropionyl substituted
  DISULFID (1)..(7)
  MOD_RES (4)..(4): D form
  MOD_RES (7)..(7): AMIDATION
CEHFRWC

SEQ ID NO: 44 - protein, 7 aa, Artificial
  Synthetic construct
  MOD_RES (1)..(1): acetyl-diaminobutyryl substituted
  DISULFID (1)..(7)
  MOD_RES (4)..(4): D form
  MOD_RES (7)..(7): AMIDATION
CEHFRWC

SEQ ID NO: 45 - protein, 8 aa, Artificial
  Synthetic construct
  DISULFID (2)..(8)
  MOD_RES (5)..(5): D form
RCEHFRWC

SEQ ID NO: 46 - protein, 8 aa, Artificial
  Synthetic construct
  MOD_RES (1)..(1): D form
  DISULFID (2)..(8)
  MOD_RES (5)..(5): D form
  MOD_RES (8)..(8): AMIDATION
RCEHFRWC

SEQ ID NO: 47 - protein, 8 aa, Artificial
  Synthetic construct
  MOD_RES (1)..(1): ACETYLATION
  MOD_RES (1)..(1): D form
  DISULFID (2)..(8)
  MOD_RES (8)..(8): AMIDATION
RCEHFRWC

SEQ ID NO: 48 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
RCEHFRWC

SEQ ID NO: 49 - protein, 8 aa, Artificial
  Synthetic construct
  MOD_RES (1)..(1): ACETYLATION
  DISULFID (2)..(8)
  MOD_RES (5)..(5): D form
RCEHFRWC

SEQ ID NO: 50 - protein, 8 aa, Artificial
  Synthetic construct
  MOD_RES (1)..(1): ACETYLATION
  DISULFID (2)..(8)
  MOD_RES (5)..(5): 4-chloro substituted, D form
  MOD_RES (8)..(8): AMIDATION
RCEHFRWC

SEQ ID NO: 51 - protein, 8 aa, Artificial
  Synthetic construct
  MOD_RES (1)..(1): ACETYLATION
  DISULFID (2)..(8)
  MOD_RES (4)..(4): 1-methyl substituted
  MOD_RES (5)..(5): D form
  MOD_RES (8)..(8): AMIDATION
RCEHFRWC

SEQ ID NO: 52 - protein, 8 aa, Artificial
  Synthetic construct
  MOD_RES (1)..(1): ACETYLATION
  MOD_RES (1)..(1): D form
  DISULFID (2)..(8)
  MOD_RES (5)..(5): D form
  MOD_RES (8)..(8): AMIDATION
RCEHFRWC

SEQ ID NO: 53 - protein, 8 aa, Artificial
  Synthetic construct
  MOD_RES (1)..(1): ACETYLATION
  MOD_RES (1)..(1): D form
  DISULFID (2)..(8)
  MOD_RES (5)..(5): D form
RCEHFRWC

SEQ ID NO: 54 - protein, 8 aa, Artificial
  Synthetic construct
  MOD_RES (1)..(1): ACETYLATION
  MISC_FEATURE (1)..(1): Xaa = homoarginine
  DISULFID (2)..(8)
  MOD_RES (5)..(5): D form
  MOD_RES (8)..(8): AMIDATION
XCEHFRWC

SEQ ID NO: 55 - protein, 8 aa, Artificial
  Synthetic construct
  MOD_RES (1)..(1): ACETYLATION
  MISC_FEATURE (1)..(1): Xaa = citrulline
  DISULFID (2)..(8)
  MOD_RES (5)..(5): D form
  MOD_RES (8)..(8): AMIDATION
XCEHFRWC

SEQ ID NO: 56 - protein, 8 aa, Artificial
  Synthetic construct
  MOD_RES (1)..(1): ACETYLATION
  MISC_FEATURE (1)..(1): Xaa = citrulline
  DISULFID (2)..(8)
  MOD_RES (4)..(4): 1-methyl substituted
  MOD_RES (5)..(5): D form
  MOD_RES (8)..(8): AMIDATION
XCEHFRWC

SEQ ID NO: 57 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
LCEHFRWC

SEQ ID NO: 58 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
KCEHFRWC

SEQ ID NO: 59 - protein, 8 aa, Artificial
  Synthetic construct
  MOD_RES (1)..(1): ACETYLATION
  MISC_FEATURE (1)..(1): Xaa = N(epsilon)-isopropyl lysine
  DISULFID (2)..(8)
  MOD_RES (5)..(5): D form
  MOD_RES (8)..(8): AMIDATION
XCEHFRWC

SEQ ID NO: 60 - protein, 8 aa, Artificial
  Synthetic construct
  MOD_RES (1)..(1): ACETYLATION
  MISC_FEATURE (1)..(1): Xaa = norleucine
  DISULFID (2)..(8)
  MOD_RES (5)..(5): D form
  MOD_RES (8)..(8): AMIDATION
XCEHFRWC

SEQ ID NO: 61 - protein, 10 aa, Artificial
  Synthetic construct
  MOD_RES (1)..(1): ACETYLATION
  MISC_FEATURE (1)..(1): Xaa = norleucine
  DISULFID (2)..(8)
  MOD_RES (5)..(5): D form
  MOD_RES (10)..(10): AMIDATION
XCEHFRWCSP

SEQ ID NO: 62 - protein, 8 aa, Artificial
  Synthetic construct
  MOD_RES (1)..(1): ACETYLATION
  MISC_FEATURE (1)..(1): Xaa = Ornithine
  DISULFID (2)..(8)
  MOD_RES (5)..(5): D form
  MOD_RES (8)..(8): AMIDATION
XCEHFRWC

SEQ ID NO: 63 - protein, 8 aa, Artificial
  Synthetic construct
  MOD_RES (1)..(1): ACETYLATION
  DISULFID (2)..(8)
  MOD_RES (5)..(5): D form
  MOD_RES (8)..(8): AMIDATION
VCEHFRWC

SEQ ID NO: 64 - protein, 8 aa, Artificial
  Synthetic construct
  MOD_RES (1)..(1): N-(2-naphthalenesulfonyl) substituted, D form
  DISULFID (2)..(8)
  MOD_RES (5)..(5): D form
  MOD_RES (8)..(8): AMIDATION
RCEHFRWC

SEQ ID NO: 65 - protein, 8 aa, Artificial
  Synthetic construct
  MOD_RES (1)..(1): N-(2-naphthalenesulfonylamino-4-oxo-butyryl) substituted
  MOD_RES (1)..(1): D form
  DISULFID (2)..(8)
  MOD_RES (5)..(5): D form
  MOD_RES (8)..(8): AMIDATION
RCEHFRWC

SEQ ID NO: 66 - protein, 8 aa, Artificial
  Synthetic construct
  MOD_RES (1)..(1): 3-(4-hydroxyphenyl)propionyl substituted
  DISULFID (2)..(8)
  MOD_RES (5)..(5): D form
  MOD_RES (8)..(8): AMIDATION
RCEHFRWC

SEQ ID NO: 67 - protein, 8 aa, Artificial
  Synthetic construct
  MOD_RES (1)..(1): 3-(4-methylbenzoyl)propionyl) substituted
  DISULFID (2)..(8)
  MOD_RES (5)..(5): D form
  MOD_RES (8)..(8): AMIDATION
RCEHFRWC

SEQ ID NO: 68 - protein, 9 aa, Artificial
  Synthetic construct
  DISULFID (3)..(9)
  MOD_RES (6)..(6): D form
  MOD_RES (9)..(9): AMIDATION
YRCEHFRWC

SEQ ID NO: 69 - protein, 9 aa, Artificial
  Synthetic construct
  DISULFID (3)..(9)
  MOD_RES (6)..(6): D form
YRCEHFRWC

SEQ ID NO: 70 - protein, 9 aa, Artificial
  Synthetic construct
  DISULFID (3)..(9)
  MOD_RES (6)..(6): D form
  MOD_RES (9)..(9): NH-(CH2)6-NH2 substituted
YRCEHFRWC

SEQ ID NO: 71 - protein, 10 aa, Artificial
  Synthetic construct
  DISULFID (3)..(9)
  MOD_RES (6)..(6): D form
  MOD_RES (10)..(10): AMIDATION
YRCEHFRWCE

SEQ ID NO: 72 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YRCEHFRWC

SEQ ID NO: 73 - protein, 9 aa, Artificial
  Synthetic construct
  MOD_RES (1)..(1): ACETYLATION
  DISULFID (3)..(9)
  MOD_RES (6)..(6): D form
YRCEHFRWC

SEQ ID NO: 74 - protein, 9 aa, Artificial
  Synthetic construct
  MOD_RES (1)..(1): N-succinyl substituted
  DISULFID (3)..(9)
  MOD_RES (6)..(6): D form
  MOD_RES (9)..(9): AMIDATION
YRCEHFRWC

SEQ ID NO: 75 - protein, 9 aa, Artificial
  Synthetic construct
  MOD_RES (1)..(1): N-glutaryl substituted
  DISULFID (3)..(9)
  MOD_RES (6)..(6): D form
  MOD_RES (9)..(9): AMIDATION
YRCEHFRWC

SEQ ID NO: 76 - protein, 9 aa, Artificial
  Synthetic construct
  MOD_RES (1)..(1): N-glutaryl substituted
  DISULFID (3)..(9)
  MOD_RES (6)..(6): D form
YRCEHFRWC

SEQ ID NO: 77 - protein, 9 aa, Artificial
  Synthetic construct
  MOD_RES (1)..(1): gluconoyl substituted
  DISULFID (3)..(9)
  MOD_RES (6)..(6): D form
  MOD_RES (9)..(9): AMIDATION
YRCEHFRWC

SEQ ID NO: 78 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): Xaa = Cys reduced from amino acid to amino alcohol
YRCEHFRWX

SEQ ID NO: 79 - protein, 9 aa, Artificial
  Synthetic construct
  MOD_RES (1)..(1): ACETYLATION
  MOD_RES (2)..(2): D form
  DISULFID (3)..(9)
  MOD_RES (6)..(6): D form
  MOD_RES (9)..(9): AMIDATION
YRCEHFRWC

SEQ ID NO: 80 - protein, 9 aa, Artificial
  Synthetic construct
  MOD_RES (1)..(1): ACETYLATION
  DISULFID (3)..(9)
  MOD_RES (3)..(3): D form
  MOD_RES (6)..(6): D form
  MOD_RES (9)..(9): AMIDATION
YRCEHFRWC

SEQ ID NO: 81 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): D form
  MOD_RES (9)..(9): AMIDATION
YRCEHFRWC

SEQ ID NO: 82 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): D form
  MOD_RES (9)..(9): AMIDATION
YRCEHFRWC

SEQ ID NO: 83 - protein, 9 aa, Artificial
  Synthetic construct
  MOD_RES (1)..(1): ACETYLATION
  DISULFID (3)..(9)
  MOD_RES (6)..(6): 4-fluoro substituted, D form
  MOD_RES (9)..(9): AMIDATION
YRCEHFRWC

SEQ ID NO: 84 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-fluoro substituted, D form
  MOD_RES (9)..(9): AMIDATION
YRCEHFRWC

SEQ ID NO: 85 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-fluoro substituted, D form
  MOD_RES (9)..(9): AMIDATION
YRCEHFRWC

SEQ ID NO: 86 - protein, 9 aa, Artificial
  Synthetic construct
  MOD_RES (1)..(1): ACETYLATION
  DISULFID (3)..(9)
  MOD_RES (6)..(6): 4-chloro substituted, D form
  MOD_RES (9)..(9): AMIDATION
YRCEHFRWC

SEQ ID NO: 87 - protein, 8 aa, Artificial
  Synthetic construct
  MOD_RES (1)..(1): ACETYLATION
  DISULFID (2)..(8)
  MOD_RES (4)..(4): 1-methyl substituted
  MOD_RES (5)..(5): 4-chloro substituted, D form
  MOD_RES (8)..(8): AMIDATION
RCEHFRWC

SEQ ID NO: 88 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-chloro substituted, D form
  MOD_RES (9)..(9): AMIDATION
YRCEHFRWC

SEQ ID NO: 89 - protein, 9 aa, Artificial
  Synthetic construct
  MOD_RES (1)..(1): ACETYLATION
  DISULFID (3)..(9)
  MOD_RES (6)..(6): 4-bromo substituted, D form
  MOD_RES (9)..(9): AMIDATION
YRCEHFRWC

SEQ ID NO: 90 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-bromo substituted, D form
  MOD_RES (9)..(9): AMIDATION
YRCEHFRWC

SEQ ID NO: 91 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-bromo substituted, D form
  MOD_RES (9)..(9): AMIDATION
YRCEHFRWC

SEQ ID NO: 92 - protein, 9 aa, Artificial
  Synthetic construct
  MOD_RES (1)..(1): ACETYLATION
  DISULFID (3)..(9)
  MOD_RES (6)..(6): 4-methyl substituted, D form
  MOD_RES (9)..(9): AMIDATION
YRCEHFRWC

SEQ ID NO: 93 - protein, 9 aa, Artificial
  Synthetic construct
  MOD_RES (1)..(1): ACETYLATION
  DISULFID (3)..(9)
  MOD_RES (6)..(6): 4-methoxy substituted, D form
  MOD_RES (9)..(9): AMIDATION
YRCEHFRWC

SEQ ID NO: 94 - protein, 9 aa, Artificial
  Synthetic construct
  MOD_RES (1)..(1): ACETYLATION
  DISULFID (3)..(9)
  MOD_RES (5)..(5): 1-methyl substituted
  MOD_RES (6)..(6): 4-methoxy substituted, D form
  MOD_RES (9)..(9): AMIDATION
YRCEHFRWC

SEQ ID NO: 95 - protein, 9 aa, Artificial
  Synthetic construct
  MOD_RES (1)..(1): ACETYLATION
  DISULFID (3)..(9)
  MOD_RES (5)..(5): 1-methyl substituted, D form
  MOD_RES (6)..(6): 4-methoxy substituted, D form
  MOD_RES (9)..(9): AMIDATION
YRCEHFRWC

SEQ ID NO: 96 - protein, 9 aa, Artificial
  Synthetic construct
  MOD_RES (1)..(1): ACETYLATION
  DISULFID (3)..(9)
  MOD_RES (5)..(5): 3-methyl substituted
  MOD_RES (6)..(6): D form
  MOD_RES (9)..(9): AMIDATION
YRCEHFRWC

SEQ ID NO: 97 - protein, 9 aa, Artificial
  Synthetic construct
  MOD_RES (1)..(1): ACETYLATION
  DISULFID (3)..(9)
  MOD_RES (5)..(5): 5-methyl substituted
  MOD_RES (6)..(6): D form
  MOD_RES (9)..(9): AMIDATION
YRCEHFRWC

SEQ ID NO: 98 - protein, 9 aa, Artificial
  Synthetic construct
  MOD_RES (1)..(1): ACETYLATION
  DISULFID (3)..(9)
  MOD_RES (5)..(5): 5-methyl substituted, D form
  MOD_RES (6)..(6): D form
  MOD_RES (9)..(9): AMIDATION
YRCEHFRWC

SEQ ID NO: 99 - protein, 9 aa, Artificial
  Synthetic construct
  MOD_RES (1)..(1): ACETYLATION
  DISULFID (3)..(9)
  MOD_RES (5)..(5): 1-benzyl substituted
  MOD_RES (6)..(6): D form
  MOD_RES (9)..(9): AMIDATION
YRCEHFRWC

SEQ ID NO: 100 - protein, 9 aa, Artificial
  Synthetic construct
  MOD_RES (1)..(1): ACETYLATION
  DISULFID (3)..(9)
  MOD_RES (5)..(5): 1-benzyl substituted, D form
  MOD_RES (6)..(6): D form
  MOD_RES (9)..(9): AMIDATION
YRCEHFRWC

SEQ ID NO: 101 - protein, 9 aa, Artificial
  Synthetic construct
  MOD_RES (1)..(1): ACETYLATION
  DISULFID (3)..(9)
  MOD_RES (5)..(5): 1-benzyloxymethyl substituted
  MOD_RES (6)..(6): D form
  MOD_RES (9)..(9): AMIDATION
YRCEHFRWC

SEQ ID NO: 102 - protein, 9 aa, Artificial
  Synthetic construct
  MOD_RES (1)..(1): ACETYLATION
  DISULFID (3)..(9)
  MOD_RES (5)..(5): 1-pyrazolyl substituted
  MOD_RES (6)..(6): D form
  MOD_RES (9)..(9): AMIDATION
YRCEAFRWC

SEQ ID NO: 103 - protein, 9 aa, Artificial
  Synthetic construct
  MOD_RES (1)..(1): ACETYLATION
  DISULFID (3)..(9)
  MOD_RES (5)..(5): 4-phenyl-1H-imidazol-2-yl substituted
  MOD_RES (6)..(6): D form
  MOD_RES (9)..(9): AMIDATION
YRCEAFRWC

SEQ ID NO: 104 - protein, 9 aa, Artificial
  Synthetic construct
  MOD_RES (1)..(1): ACETYLATION
  DISULFID (3)..(9)
  MOD_RES (5)..(5): 4-phenyl-1H-imidazol-2-yl substituted, D form
  MOD_RES (6)..(6): D form
  MOD_RES (9)..(9): AMIDATION
YRCEAFRWC

SEQ ID NO: 105 - protein, 9 aa, Artificial
  Synthetic construct
  MOD_RES (1)..(1): ACETYLATION
  DISULFID (3)..(9)
  MOD_RES (5)..(5): 2-pyrazine substituted
  MOD_RES (6)..(6): D form
  MOD_RES (9)..(9): AMIDATION
YRCEAFRWC

SEQ ID NO: 106 - protein, 9 aa, Artificial
  Synthetic construct
  MOD_RES (1)..(1): ACETYLATION
  DISULFID (3)..(9)
  MOD_RES (5)..(5): beta-(1,2,4-triazol-3-yl) substituted
  MOD_RES (6)..(6): D form
  MOD_RES (9)..(9): AMIDATION
YRCEAFRWC

SEQ ID NO: 107 - protein, 9 aa, Artificial
  Synthetic construct
  MOD_RES (1)..(1): ACETYLATION
  DISULFID (3)..(9)
  MOD_RES (5)..(5): beta-(1,2,4-triazol-3-yl) substituted, D form
  MOD_RES (6)..(6): D form
  MOD_RES (9)..(9): AMIDATION
YRCEAFRWC

SEQ ID NO: 108 - protein, 9 aa, Artificial
  Synthetic construct
  MOD_RES (1)..(1): ACETYLATION
  DISULFID (3)..(9)
  MOD_RES (5)..(5): beta-((1-benzyl)-1,2,4-triazol-3-yl) substituted
  MOD_RES (6)..(6): D form
  MOD_RES (9)..(9): AMIDATION
YRCEAFRWC

SEQ ID NO: 109 - protein, 9 aa, Artificial
  Synthetic construct
  MOD_RES (1)..(1): ACETYLATION
  DISULFID (3)..(9)
  MOD_RES (5)..(5): beta-((1-benzyl)-1,2,4-triazol-3-yl) substituted, D form
  MOD_RES (6)..(6): D form
  MOD_RES (9)..(9): AMIDATION
YRCEAFRWC

SEQ ID NO: 110 - protein, 9 aa, Artificial
  Synthetic construct
  MOD_RES (1)..(1): ACETYLATION
  DISULFID (3)..(9)
  MOD_RES (5)..(5): beta-(2-furyl) substituted
  MOD_RES (6)..(6): D form
  MOD_RES (9)..(9): AMIDATION
YRCEAFRWC

SEQ ID NO: 111 - protein, 9 aa, Artificial
  Synthetic construct
  MOD_RES (1)..(1): ACETYLATION
  DISULFID (3)..(9)
  MOD_RES (5)..(5): beta-(thien-2-yl) substituted
  MOD_RES (6)..(6): D form
  MOD_RES (9)..(9): AMIDATION
YRCEAFRWC

SEQ ID NO: 112 - protein, 9 aa, Artificial
  Synthetic construct
  MOD_RES (1)..(1): ACETYLATION
  DISULFID (3)..(9)
  MOD_RES (5)..(5): beta-(1,3-thiazol-4-yl) substituted
  MOD_RES (6)..(6): D form
  MOD_RES (9)..(9): AMIDATION
YRCEAFRWC

SEQ ID NO: 113 - protein, 9 aa, Artificial
  Synthetic construct
  MOD_RES (1)..(1): ACETYLATION
  DISULFID (3)..(9)
  MOD_RES (5)..(5): beta-(pyridin-4-yl) substituted
  MOD_RES (6)..(6): D form
  MOD_RES (9)..(9): AMIDATION
YRCEAFRWC

SEQ ID NO: 114 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): glycinol substituted
YRCEHFRWC

SEQ ID NO: 115 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): 2-(2-aminoethoxy)ethanol substituted
YRCEHFRWC

SEQ ID NO: 116 - protein, 10 aa, Artificial
  Synthetic construct
  MOD_RES (1)..(1): ACETYLATION
  DISULFID (3)..(9)
  MOD_RES (6)..(6): D form
  MOD_RES (10)..(10): Xaa = Ser reduced from amino acid to amino alcohol
YRCEHFRWCX

SEQ ID NO: 117 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): NH-(CH2)6-NH2 substituted
YRCEHFRWC

SEQ ID NO: 118 - protein, 10 aa, Artificial
  Synthetic construct
  MOD_RES (1)..(1): ACETYLATION
  DISULFID (3)..(9)
  MOD_RES (6)..(6): D form
  MOD_RES (10)..(10): AMIDATION
YRCEHFRWCE

SEQ ID NO: 119 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCSP

SEQ ID NO: 120 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): Xaa = Pro reduced from amino acid to amino alcohol
YRCEHFRWCSX

SEQ ID NO: 121 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCKP

SEQ ID NO: 122 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): Xaa = Pro reduced from amino acid to amino alcohol
YRCEHFRWCKX

SEQ ID NO: 123 - protein, 11 aa, Artificial
  Synthetic construct
  MOD_RES (1)..(1): ACETYLATION
  DISULFID (3)..(9)
  MOD_RES (6)..(6): D form
  MOD_RES (11)..(11): AMIDATION
YRCEHFRWCRF

SEQ ID NO: 124 - protein, 9 aa, Artificial
  Synthetic construct
  MOD_RES (1)..(1): ACETYLATION
  MISC_FEATURE (2)..(2): Xaa = citrulline
  DISULFID (3)..(9)
  MOD_RES (6)..(6): D form
  MOD_RES (9)..(9): AMIDATION
YXCEHFRWC

SEQ ID NO: 125 - protein, 9 aa, Artificial
  Synthetic construct
  MOD_RES (1)..(1): ACETYLATION
  MISC_FEATURE (2)..(2): Xaa = citrulline
  DISULFID (3)..(9)
  MOD_RES (5)..(5): 1-methyl substituted
  MOD_RES (6)..(6): D form
  MOD_RES (9)..(9): AMIDATION
YXCEHFRWC

SEQ ID NO: 126 - protein, 9 aa, Artificial
  Synthetic construct
  MOD_RES (1)..(1): ACETYLATION
  MISC_FEATURE (2)..(2): Xaa = homoarginine
  DISULFID (3)..(9)
  MOD_RES (6)..(6): D form
  MOD_RES (9)..(9): AMIDATION
YXCEHFRWC

SEQ ID NO: 127 - protein, 9 aa, Artificial
  Synthetic construct
  MOD_RES (1)..(1): ACETYLATION
  MISC_FEATURE (2)..(2): Xaa = 1-beta-homoarginine
  DISULFID (3)..(9)
  MOD_RES (6)..(6): D form
  MOD_RES (9)..(9): AMIDATION
YXCEHFRWC

SEQ ID NO: 128 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YKCEHFRWC

SEQ ID NO: 129 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YSCEHFRWC

SEQ ID NO: 130 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
YVCEHFRWC

SEQ ID NO: 131 - protein, 9 aa, Artificial
  Synthetic construct
  MOD_RES (1)..(1): N-succinyl substituted
  DISULFID (3)..(9)
  MOD_RES (6)..(6): D form
YRCEHFRWC

SEQ ID NO: 132 - protein, 6 aa, Artificial
  Synthetic construct
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 133 - protein, 6 aa, Artificial
  Synthetic construct
  DISULFID (1)..(6)
  MISC_FEATURE (1)..(1): Xaa = homocysteine
  MOD_RES (3)..(3): D form
XHFRWC

SEQ ID NO: 134 - protein, 6 aa, Artificial
  Synthetic construct
  DISULFID (1)..(6)
  MISC_FEATURE (1)..(1): Xaa = homocysteine
  MOD_RES (3)..(3): 4-fluoro substituted, D form
  MOD_RES (6)..(6): AMIDATION
XHFRWC

SEQ ID NO: 135 - protein, 6 aa, Artificial
  Synthetic construct
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
XHFRWC

SEQ ID NO: 136 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (6)..(6): AMIDATION
XHFRWC

SEQ ID NO: 137 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 138 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
XHFRWC

SEQ ID NO: 139 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-fluoro substituted, D form
  MOD_RES (6)..(6): AMIDATION
XHFRWC

SEQ ID NO: 140 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
XHFRWC

SEQ ID NO: 141 - protein, 6 aa, Artificial
  Synthetic construct
  MOD_RES (1)..(1): N-cyclopropanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 142 - protein, 6 aa, Artificial
  Synthetic construct
  MOD_RES (1)..(1): N-cyclobutanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 143 - protein, 6 aa, Artificial
  Synthetic construct
  MOD_RES (1)..(1): N-cyclopentanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 144 - protein, 6 aa, Artificial
  Synthetic construct
  MOD_RES (1)..(1): N-cyclohexanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 145 - protein, 6 aa, Artificial
  Synthetic construct
  MOD_RES (1)..(1): N-hexanoyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 146 - protein, 6 aa, Artificial
  Synthetic construct
  MOD_RES (1)..(1): N-benzoyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 147 - protein, 6 aa, Artificial
  Synthetic construct
  MOD_RES (1)..(1): 4-phenylbutyrylsubstituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 148 - protein, 6 aa, Artificial
  Synthetic construct
  MOD_RES (1)..(1): 3-guanidinopropionyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 149 - protein, 6 aa, Artificial
  Synthetic construct
  MOD_RES (1)..(1): 5-guanidinovaleryl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 150 - protein, 6 aa, Artificial
  Synthetic construct
  MOD_RES (1)..(1): N-phenylsulfonyl substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 151 - protein, 6 aa, Artificial
  Synthetic construct
  MOD_RES (1)..(1): N-(2-naphthalenesulfonyl) substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 152 - protein, 6 aa, Artificial
  Synthetic construct
  MOD_RES (1)..(1): N-(4-phenylsulfonamido-4-oxo-butyryl) substituted
  DISULFID (1)..(6)
  MISC_FEATURE (1)..(1): Xaa = homocysteine
  MOD_RES (3)..(3): D form
  MOD_RES (6)..(6): AMIDATION
XHFRWC

SEQ ID NO: 153 - protein, 7 aa, Artificial
  Synthetic construct
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 154 - protein, 7 aa, Artificial
  Synthetic construct
  MOD_RES (1)..(1): D form
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 155 - protein, 7 aa, Artificial
  Synthetic construct
  DISULFID (2)..(7)
  MISC_FEATURE (2)..(2): Xaa = homocysteine
  MOD_RES (4)..(4): D form
RXHFRWC

SEQ ID NO: 156 - protein, 7 aa, Artificial
  Synthetic construct
  DISULFID (2)..(7)
  MISC_FEATURE (2)..(2): Xaa = homocysteine
  MISC_FEATURE (3)..(3): 1-methyl substituted
  MOD_RES (4)..(4): D form
  MOD_RES (7)..(7): AMIDATION
RXHFRWC

SEQ ID NO: 157 - protein, 7 aa, Artificial
  Synthetic construct
  DISULFID (2)..(7)
  MISC_FEATURE (2)..(2): Xaa = homocysteine
  MOD_RES (3)..(3): 1-methyl substituted, D form
  MOD_RES (4)..(4): D form
  MOD_RES (7)..(7): AMIDATION
RXHFRWC

SEQ ID NO: 158 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
  MOD_RES (7)..(7): AMIDATION
RXHFRWC

SEQ ID NO: 159 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
RXHFRWC

SEQ ID NO: 160 - protein, 7 aa, Artificial
  Synthetic construct
  MOD_RES (1)..(1): ACETYLATION
  MISC_FEATURE (1)..(1): Xaa = norleucine
  DISULFID (2)..(7)
  MISC_FEATURE (2)..(2): Xaa = homocysteine
  MOD_RES (4)..(4): D form
  MOD_RES (7)..(7): AMIDATION
XXHFRWC

SEQ ID NO: 161 - protein, 7 aa, Artificial
  Synthetic construct
  MOD_RES (1)..(1): phenylsulfonyl substituted
  DISULFID (2)..(7)
  MISC_FEATURE (2)..(2): Xaa = homocysteine
  MOD_RES (4)..(4): D form
  MOD_RES (7)..(7): AMIDATION
GXHFRWC

SEQ ID NO: 162 - protein, 8 aa, Artificial
  Synthetic construct
  DISULFID (3)..(8)
  MISC_FEATURE (3)..(3): Xaa = homocysteine
  MOD_RES (5)..(5): D form
  MOD_RES (8)..(8): AMIDATION
YRXHFRWC

SEQ ID NO: 163 - protein, 8 aa, Artificial
  Synthetic construct
  DISULFID (3)..(8)
  MISC_FEATURE (3)..(3): Xaa = homocysteine
  MOD_RES (5)..(5): D form
YRXHFRWC

SEQ ID NO: 164 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
  MOD_RES (8)..(8): AMIDATION
YRXHFRWC

SEQ ID NO: 165 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
YRXHFRWC

SEQ ID NO: 166 - protein, 9 aa, Artificial
  Synthetic construct
  MOD_RES (1)..(1): ACETYLATION
  DISULFID (3)..(9)
  MISC_FEATURE (3)..(3): Xaa = homocysteine
  MOD_RES (6)..(6): D form
  MOD_RES (9)..(9): AMIDATION
YRXEHFRWC

SEQ ID NO: 167 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): beta-cyclohexyl substituted, D form
  MOD_RES (6)..(6): AMIDATION
XHARWC

SEQ ID NO: 168 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 169 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): 4-chloro substituted, D form
  MOD_RES (6)..(6): AMIDATION
  MISC_FEATURE (6)..(6): Xaa = penicillamine
XHFRWX

SEQ ID NO: 170 - protein, 6 aa, Artificial
  Synthetic construct
  MOD_RES (1)..(1): N-hexanoyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 171 - protein, 6 aa, Artificial
  Synthetic construct
  MOD_RES (1)..(1): N-cyclopentanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 172 - protein, 6 aa, Artificial
  Synthetic construct
  MOD_RES (1)..(1): N-cyclohexanecarbonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 173 - protein, 6 aa, Artificial
  Synthetic construct
  MOD_RES (1)..(1): N-benzoyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 174 - protein, 6 aa, Artificial
  Synthetic construct
  MOD_RES (1)..(1): 4-phenylbutyryl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 175 - protein, 6 aa, Artificial
  Synthetic construct
  MOD_RES (1)..(1): N-phenylsulfonyl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 176 - protein, 6 aa, Artificial
  Synthetic construct
  MOD_RES (1)..(1): (4-benzenesulfonamide)butyryl substituted
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = penicillamine
XHFRWX

SEQ ID NO: 177 - protein, 7 aa, Artificial
  Synthetic construct
  MOD_RES (1)..(1): ACETYLATION
  MISC_FEATURE (1)..(1): Xaa = norleucine
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = penicillamine
XXHFRWX

SEQ ID NO: 178 - protein, 7 aa, Artificial
  Synthetic construct
  MOD_RES (1)..(1): N-phenylsulfonyl substituted
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = penicillamine
GXHFRWX

SEQ ID NO: 179 - protein, 6 aa, Artificial
  Synthetic construct
  MISC_FEATURE (1)..(1): Xaa = desamino Cys
  DISULFID (1)..(6)
  MOD_RES (3)..(3): D form
  MOD_RES (6)..(6): AMIDATION
  MISC_FEATURE (6)..(6): Xaa = homocysteine
XHFRWX

SEQ ID NO: 180 - protein, 6 aa, Artificial
  Synthetic construct
  DISULFID (1)..(6)
  MOD_RES (3)..(3): D form
  MOD_RES (6)..(6): AMIDATION
  MISC_FEATURE (6)..(6): Xaa = homocysteine
CHFRWX

SEQ ID NO: 181 - protein, 6 aa, Artificial
  Synthetic construct
  DISULFID (1)..(6)
  MOD_RES (3)..(3): 4-fluoro substituted, D form
  MOD_RES (6)..(6): AMIDATION
  MISC_FEATURE (6)..(6): Xaa = homocysteine
CHFRWX

SEQ ID NO: 182 - protein, 6 aa, Artificial
  Synthetic construct
  DISULFID (1)..(6)
  MOD_RES (3)..(3): 4-chloro substituted, D form
  MOD_RES (6)..(6): AMIDATION
  MISC_FEATURE (6)..(6): Xaa = homocysteine
CHFRWX

SEQ ID NO: 183 - protein, 6 aa, Artificial
  Synthetic construct
  MOD_RES (1)..(1): ACETYLATION
  DISULFID (1)..(6)
  MOD_RES (3)..(3): D form
  MOD_RES (6)..(6): AMIDATION
  MISC_FEATURE (6)..(6): Xaa = homocysteine
CHFRWX

SEQ ID NO: 184 - protein, 6 aa, Artificial
  Synthetic construct
  MOD_RES (1)..(1): ACETYLATION
  DISULFID (1)..(6)
  MOD_RES (3)..(3): 4-fluoro substituted, D form
  MOD_RES (6)..(6): AMIDATION
  MISC_FEATURE (6)..(6): Xaa = homocysteine
CHFRWX

SEQ ID NO: 185 - protein, 6 aa, Artificial
  Synthetic construct
  MOD_RES (1)..(1): ACETYLATION
  DISULFID (1)..(6)
  MOD_RES (3)..(3): 4-chloro substituted, D form
  MOD_RES (6)..(6): AMIDATION
  MISC_FEATURE (6)..(6): Xaa = homocysteine
CHFRWX

SEQ ID NO: 186 - protein, 7 aa, Artificial
  Synthetic construct
  DISULFID (2)..(7)
  MOD_RES (4)..(4): D form
  MOD_RES (7)..(7): AMIDATION
  MISC_FEATURE (7)..(7): Xaa = homocysteine
RCHFRWX

SEQ ID NO: 187 - protein, 7 aa, Artificial
  Synthetic construct
  DISULFID (2)..(7)
  MOD_RES (4)..(4): 4-fluoro substituted, D form
  MOD_RES (7)..(7): AMIDATION
  MISC_FEATURE (7)..(7): Xaa = homocysteine
RCHFRWX

SEQ ID NO: 188 - protein, 7 aa, Artificial
  Synthetic construct
  DISULFID (2)..(7)
  MOD_RES (4)..(4): 4-chloro substituted, D form
  MOD_RES (7)..(7): AMIDATION
  MISC_FEATURE (7)..(7): Xaa = homocysteine
RCHFRWX

SEQ ID NO: 189 - protein, 7 aa, Artificial
  Synthetic construct
  MOD_RES (1)..(1): ACETYLATION
  DISULFID (2)..(7)
  MOD_RES (4)..(4): D form
  MOD_RES (7)..(7): AMIDATION
  MISC_FEATURE (7)..(7): Xaa = homocysteine
RCHFRWX

SEQ ID NO: 190 - protein, 7 aa, Artificial
  Synthetic construct
  MOD_RES (1)..(1): ACETYLATION
  DISULFID (2)..(7)
  MOD_RES (4)..(4): 4-fluoro substituted, D form
  MOD_RES (7)..(7): AMIDATION
  MISC_FEATURE (7)..(7): Xaa = homocysteine
RCHFRWX

SEQ ID NO: 191 - protein, 7 aa, Artificial
  Synthetic construct
  MOD_RES (1)..(1): ACETYLATION
  DISULFID (2)..(7)
  MOD_RES (4)..(4): 4-chloro substituted, D form
  MOD_RES (7)..(7): AMIDATION
  MISC_FEATURE (7)..(7): Xaa = homocysteine
RCHFRWX

SEQ ID NO: 192 - protein, 9 aa, Artificial
  Synthetic construct
  MOD_RES (1)..(1): ACETYLATION
  DISULFID (3)..(9)
  MOD_RES (6)..(6): D form
  MOD_RES (9)..(9): AMIDATION
  MISC_FEATURE (9)..(9): Xaa = homocysteine
YRCEHFRWX

SEQ ID NO: 193 - protein, 6 aa, Artificial
  Synthetic construct
  MOD_RES (1)..(1): ACETYLATION
  DISULFID (1)..(6)
  MISC_FEATURE (1)..(1): Xaa = homocysteine
  MOD_RES (3)..(3): D form
  MOD_RES (6)..(6): AMIDATION
  MISC_FEATURE (6)..(6): Xaa = homocysteine
XHFRWX

SEQ ID NO: 194 - protein, 7 aa, Artificial
  Synthetic construct
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = homocysteine
RXHFRWX

SEQ ID NO: 195 - protein, 7 aa, Artificial
  Synthetic construct
  MOD_RES (1)..(1): ACETYLATION
  DISULFID (2)..(7)
  MISC_FEATURE (2)..(2): Xaa = homocysteine
  MOD_RES (4)..(4): D form
  MOD_RES (7)..(7): AMIDATION
  MISC_FEATURE (7)..(7): Xaa = homocysteine
RXHFRWX

SEQ ID NO: 196 - protein, 8 aa, Artificial
  Synthetic construct
  MOD_RES (1)..(1): ACETYLATION
  DISULFID (3)..(8)
  MISC_FEATURE (3)..(3): Xaa = homocysteine
  MOD_RES (5)..(5): D form
  MOD_RES (8)..(8): AMIDATION
  MISC_FEATURE (8)..(8): Xaa = homocysteine
YRXHFRWX

SEQ ID NO: 197 - protein, 9 aa, Artificial
  Synthetic construct
  MOD_RES (1)..(1): ACETYLATION
  DISULFID (3)..(9)
  MISC_FEATURE (3)..(3): Xaa = homocysteine
  MOD_RES (6)..(6): D form
  MOD_RES (9)..(9): AMIDATION
  MISC_FEATURE (9)..(9): Xaa = homocysteine
YRXEHFRWX

SEQ ID NO: 198 - protein, 6 aa, Artificial
  Synthetic construct
  MOD_RES (1)..(1): ACETYLATION
  DISULFID (1)..(6): S-CH2-S linkage
  MOD_RES (3)..(3): D form
  MOD_RES (6)..(6): AMIDATION
CHFRWC

SEQ ID NO: 199 - protein, 9 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Cya-Arg, Tyr-homoarginine, Tyr-1-beta- homoarginine, Tyr-Lys, Tyr-Ser, or Tyr-Val
  DISULFID (2)..(8): S-S or S-CH2-S disulfide bridge
  MISC_FEATURE (2)..(2): Xaa = Cys, homocysteine, or desamino-cysteine; may be D or L form
  MISC_FEATURE (3)..(3): Xaa = Glu, Gln, Asp, Asn, Ala, Gly, Thr, Ser, Pro, Met, Ile, Val, Arg, His, Tyr, Trp, Phe, Lys, Leu, cysteic acid, or is absent
  MISC_FEATURE (4)..(4): Xaa = His, modified His, or modified Ala; D or L form
  MISC_FEATURE (5)..(5): Xaa = Phe, modified Phe, or modified Ala; D or L form
  MISC_FEATURE (6)..(6): Xaa = Arg or modified Arg; D or L form
  MISC_FEATURE (8)..(8): Xaa = Cys, homocysteine, or modified cysteine or homocysteine (such as amide, alcohol, or penicillamine)
  MISC_FEATURE (9)..(9): Xaa = Ser-Pro-NH2, Lys-Pro-NH2, Ser-OH, Ser-Pro-OH, Lys-OH, Ser alcohol, Ser-Pro alcohol, Arg-Phe-NH2, Glu-NH2, or is absent
XXXXXXWXX

SEQ ID NO: 200 - protein, 8 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Cya-Arg, Tyr-homoarginine, Tyr-1-beta- homoarginine, Tyr-Lys, Tyr-Ser, or Tyr-Val
  DISULFID (2)..(8)
  MISC_FEATURE (2)..(2): Xaa = Cys or homocysteine
  MISC_FEATURE (3)..(3): Xaa = Glu, Gln, Asp, Asn, Ala, Gly, Thr, Ser, Pro, Met, Ile, Val, Arg, His, Tyr, Trp, Phe, Lys, Leu, cysteic acid, or is absent
  MOD_RES (4)..(4): His may be optionally substituted, D or L form
  MOD_RES (5)..(5): Phe may be optionally substituted, D or L form
  MISC_FEATURE (8)..(8): Xaa = Cys, homocysteine, or modified cysteine or homocysteine such as amide
  MISC_FEATURE (9)..(9): Xaa = Ser-Pro-NH2, Lys-Pro-NH2, Ser-OH, Ser-Pro-OH, Lys-OH, Ser alcohol, Ser-Pro alcohol, Arg-Phe-NH2, Glu-NH2, or is absent
XXXHFRXX

SEQ ID NO: 201 - protein, 9 aa, Artificial
  Synthetic construct
  MISC_FEATURE (1)..(1): Xaa = Arg, Tyr-Arg, Tyr-beta-Arg, or is absent
  MISC_FEATURE (1)..(1): Xaa = a modified amino acid including Arg, citrulline, homoarginine, Leu, Lys, N-isopropyl-Lys, norleucine, ornithine, or Val
  MISC_FEATURE (1)..(1): Xaa = a modified group including Tyr-Arg, Tyr-citrulline, Tyr-homoarginine, Tyr-1-beta-homoarginine, Tyr-Lys, Tyr-Ser, or Tyr-Val
  DISULFID (2)..(8)
  MISC_FEATURE (2)..(2): Xaa = Cys or homocysteine
  MISC_FEATURE (3)..(3): Xaa = Glu, Gln, Asp, Asn, Ala, Gly, Thr, Ser, Pro, Met, Ile, Val, Arg, His, Tyr, Trp, Phe, or is absent
  MOD_RES (4)..(4): His may be optionally substituted, D or L form
  MOD_RES (5)..(5): Phe may be optionally substituted, D or L form
  MISC_FEATURE (8)..(8): Xaa = Cys, homocysteine, or modified cysteine or homocysteine such as amide
  MISC_FEATURE (9)..(9): Xaa = Ser-Pro-NH2, Lys-Pro-NH2, Ser-OH, Ser-Pro-OH, Lys-Pro-OH, Arg-Phe-NH2, Glu-NH2, or is absent
XXXHFRWXX